(19) United States
(12) Patent Application Publication
(10) Pub. No.: US 2026/0249089 A1
(43) Pub. Date: Aug. 27, 2026

(54) STIMULATION CONTROL FOR ENHANCED ENERGY HARVESTING

(71) Applicant: Medtronic, Inc., Minneapolis, MN (US)

(72) Inventors: Can Cinbis, Salt Lake City, UT (US); Kevin L. Sack, Minneapolis, MN (US); Amin Karami, Ann Arbor, MI (US); Joshua S. Niesen, Circle Pines, MN (US)

(21) Appl. No.: 19/159,207

(22) PCT Filed: Feb. 14, 2024
(86) PCT No.: PCT/IB2024/051386

§ 371 Date: Aug. 22, 2025

Related U.S. Application Data
(60) Provisional application No. 63/486,860, filed on Feb. 24, 2023.

(51) Int. Cl. A61N 1/378; A61N 1/02; A61N 1/365; A61N 1/375

(52) U.S. Cl. CPC A61N 1/3785; A61N 1/025; A61N 1/36578; A61N 1/37512; A61N 1/3756

(57) ABSTRACT

Devices for energy harvesting are disclosed herein. The present disclosure concerns a device (400) comprising: an energy harvesting mechanism (422) configured to produce energy from physiological motion of a patient; one or more electrodes (402a, 402b, 402c) configured to deliver electrical stimulation to the patient; a power source (420) operably coupled to the energy harvesting mechanism and the one or more electrodes; processing circuitry (414); and a memory (418) operably coupled to the processing circuitry and storing instructions that, when executed by the processing circuitry, cause the device to perform operations comprising: determining (702) a power output of the energy harvesting mechanism; determining (704) a stimulation signal configured to adjust the physiological motion to increase the power output of the energy harvesting mechanism; delivering (706) the stimulation signal to the patient using the one or more electrodes; and charging (708) the power source using the energy harvesting mechanism.

This application claims the benefit of U.S. Provisional Patent Application Ser. No. 63/486,860, filed Feb. 24, 2023, the entire content of which is incorporated herein by reference.

### TECHNICAL FIELD

The present technology generally relates to medical devices, and in particular, to devices and methods for enhancing energy harvesting via stimulation control.

### BACKGROUND

Various types of implantable medical devices have been developed for monitoring or treating one or more conditions of a patient. For example, a cardiac pacemaker can monitor a patient's heart activity and provide therapeutic electrical stimulation to the heart via electrodes. The electrical stimulation provided by the cardiac pacemaker can include signals such as pacing pulses to address abnormal cardiac rhythms (e.g., bradycardia). Some types of cardiac pacemakers are implanted a distance from the heart and are coupled to one or more leads that extend intravascularly into the heart to position the electrodes in contact with cardiac tissue. However, the leads may be prone to fracture, which may result in unreliable or incorrect pacing, and may require replacement of the lead or even the entire pacemaker.

Some types of cardiac pacemakers are sized to be completely implanted within one of the chambers of the heart, and may include electrodes integrated with or attached to the device housing rather than leads. Such pacemakers can be less invasive than traditional pacemakers and can avoid complications associated with lead fracture. However, the relatively small size of such pacemakers may limit the types of power sources that can be incorporated into the device.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 illustrates a pacing device implanted in the heart of a patient, in accordance with embodiments of the present technology.

FIG. 2 is a perspective view of a pacing device configured in accordance with embodiments of the present technology.

FIG. 3 is a side view of another pacing device configured in accordance with embodiments of the present technology.

FIG. 4 is a schematic block diagram illustrating electronic components of a pacing device configured in accordance with embodiments of the present technology.

FIG. 5 is a side cross-sectional view of a device including an energy harvesting mechanism, in accordance with embodiments of the present technology.

FIG. 6A is a graph illustrating an example of a frequency spectrum of cardiac motion, in accordance with embodiments of the present technology.

FIG. 6B illustrates an example comparison between the frequency characteristics of an energy harvesting mechanism and the frequency spectrum of cardiac motion, in accordance with embodiments of the present technology.

FIG. 6C is a graph illustrating examples of full-time frequency spectra for cardiac motion at different heart rates, in accordance with embodiments of the present technology.

FIG. 7 is a flow diagram illustrating a method for powering an implantable device, in accordance with embodiments of the present technology.

FIG. 8 is a graph illustrating an example transfer function between a pacing rate determined using a rate-responsive pacing function and a modified pacing rate to enhance energy harvesting, in accordance with embodiments of the present technology.

FIG. 9 is a flow diagram illustrating a method for determining a stimulation signal to enhance power output of an energy harvesting mechanism, in accordance with embodiments of the present technology.

FIG. 10 is a flow diagram illustrating a method for powering an implantable device, in accordance with embodiments of the present technology.

### DETAILED DESCRIPTION

The present technology relates to devices and methods for harvesting energy using an implantable medical device. In some embodiments, for example, an implantable device includes an energy harvesting mechanism (e.g., a piezoelectric harvester) configured to produce energy from physiological motion of a patient (e.g., cardiac motion). The device can include one or more electrodes configured to deliver a stimulation signal (e.g., a cardiac pacing signal) to the patient, and a power source operably coupled to the energy harvesting mechanism and the one or more electrodes. The device can also include processing circuitry and a memory storing instructions that, when executed by the processing circuitry, cause the device to perform operations for enhancing the power output of the energy harvesting mechanism. In some embodiments, the operations include determining a power output of the energy harvesting mechanism (e.g., net power into the power source), and determining a stimulation signal configured to adjust the physiological motion to increase the power output of the energy harvesting mechanism. For instance, the device can determine a pacing rate for a cardiac pacing signal that modifies the frequency spectrum of the cardiac motion so that at least one spectral peak of the frequency spectrum matches or overlaps with a resonant frequency of the energy harvesting mechanism. Subsequently, the device can deliver the determined stimulation signal to the patient using the one or more electrodes, and can charge the power source using the energy harvesting mechanism.

The present technology can provide numerous advantages compared to conventional approaches for powering implantable devices. For example, the use of kinetic energy harvesters that produce electrical energy from physiological motion as described herein can extend the lifetime of the implantable device by allowing for recharging in situ within the patient's body. The power generated by such harvesters may be increased and/or maximized when the frequency components of the physiological motion are close to the resonant frequency or frequencies of the harvester, resulting in resonant behavior. In some embodiments, the devices herein implement an algorithm that selects appropriate parameters of a stimulation signal to adjust the physiological motion to enhance energy harvesting, while also providing a therapeutic benefit to the patient. This stimulation control-based optimization of energy harvesting can improve the longevity of the implantable device and improve safety by mitigating the risk of battery depletion.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

As used herein, the terms “vertical,” “lateral,” “upper,” and “lower” can refer to relative directions or positions of features of the embodiments disclosed herein in view of the orientation shown in the Figures. For example, “upper” or “uppermost” can refer to a feature positioned closer to the top of a page than another feature. These terms, however, should be construed broadly to include embodiments having other orientations, such as inverted or inclined orientations where top/bottom, over/under, above/below, up/down, and left/right can be interchanged depending on the orientation.

The headings provided herein are for convenience only and do not interpret the scope or meaning of the claimed present technology. Embodiments under any one heading may be used in conjunction with embodiments under any other heading.

### I. Overview of Implantable Pacing Devices

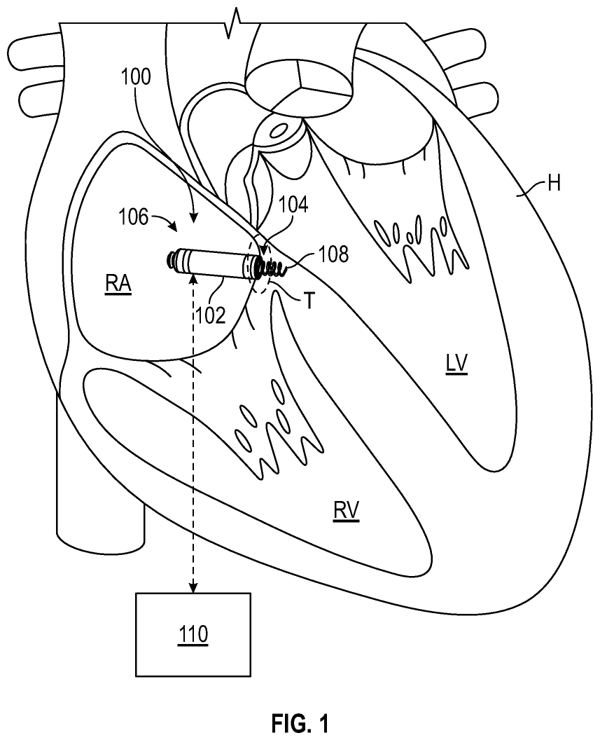

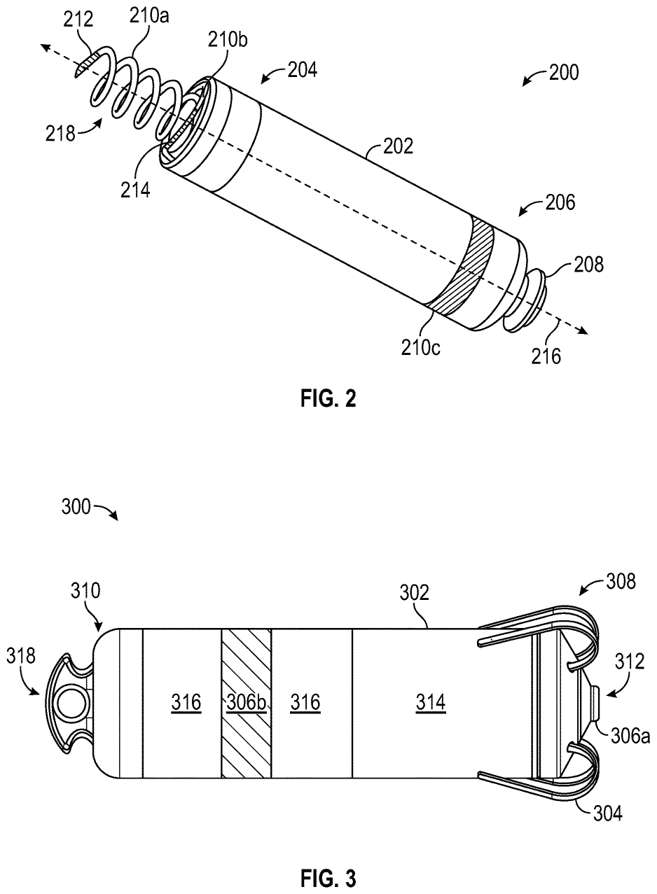

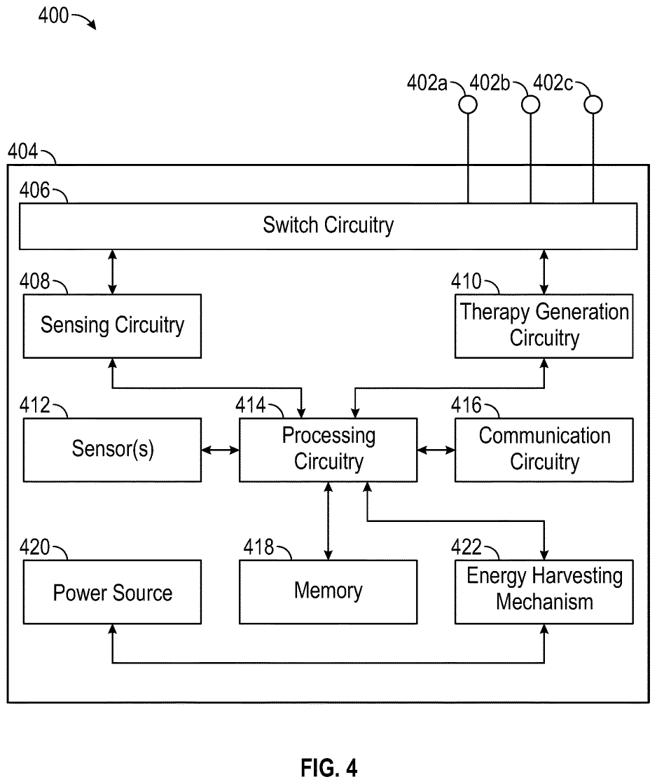

FIGS. 1-4 provide a general overview of implantable devices configured in accordance with embodiments of the present technology. Specifically, FIG. 1 illustrates a pacing device implanted in a patient's heart, FIG. 2 illustrates an example configuration for a pacing device, FIG. 3 illustrates another example configuration for a pacing device, and FIG. 4 illustrates electronic components that can be included in a pacing device. Any of the features of the embodiments of FIGS. 1-4 can be combined with each other and/or with any of the other embodiments described herein.

Referring first to FIG. 1, which illustrates a pacing device 100 implanted in the heart H of a patient, the device 100 is configured to monitor activity of the heart H and provide electrical stimulation (e.g., pacing signals) to the heart H. In some embodiments, the device 100 is a leadless intracardiac pacemaker configured to be implanted entirely within a heart chamber, such as entirely within the right atrium (RA), entirely within the right ventricle (RV), entirely within the left atrium (LA), or entirely within the left ventricle (LV). The device 100 can be implanted at any of a variety of locations to sense and/or deliver therapy to any chamber or chambers of the heart H. For example, as shown in FIG. 1, the device 100 can be a right atrial intracardiac pacemaker that is implanted in the RA of the patient's heart H in a target implant region T (e.g., the triangle of Koch). The target implant region T can lie between the bundle of His and the coronary sinus, and/or can be adjacent to the tricuspid valve. In other embodiments, the device 100 can instead be configured as a right ventricular intracardiac pacemaker that is implanted in the RV of the heart H, with the target implant region T lying along the endocardial wall at or near the apex of the RV.

The device 100 can include a housing 102 having a size and form factor suitable for transvenous delivery into the heart H via a catheter. In the illustrated embodiment, the housing 102 has an elongate shape extending from a distal portion 104 to a proximal portion 106. The housing 102 can have a generally cylindrical shape (e.g., pill-shaped or capsule-shaped), a generally prismatic shape (e.g., a rectangular prism), or any other suitable shape. The housing 102 can define an interior cavity that contains the electronic components of the device 100 (e.g., circuitry, power source, sensors).

The device 100 can include a fixation mechanism 108 to secure the device 100 to the tissue of the heart H. For example, the fixation mechanism 108 can include one or more fixation elements configured to penetrate into tissue, such as one or more tines, coils, barbs, etc. In the illustrated embodiment, the fixation mechanism 108 is coupled to and extends outwardly from the distal portion 104 of the housing 102. Accordingly, when the device 100 is implanted, the distal portion 104 can be positioned in contact with or in close proximity to the cardiac tissue, while the proximal portion 106 can be spaced apart from the cardiac tissue. In other embodiments, however, the fixation mechanism 108 can be located at a different portion of the device 100.

The device 100 also includes a plurality of electrodes configured to sense electrical activity of the heart H and/or deliver electrical therapy to the heart H. For example, the device 100 can include two, three, four, five, six, seven, eight, nine, ten, or more electrodes. Each electrode can be positioned at any suitable portion of the device 100, such as on or coupled to the housing 102 (e.g., the distal portion 104, the proximal portion 106, an intermediate location between the distal portion 104 and proximal portion 106), or on or coupled to the fixation mechanism 108. In some embodiments, the device 100 includes one or more electrodes (e.g., cathodes) that directly contact the cardiac tissue (e.g., of a single heart chamber or multiple heart chambers) to sense the activity thereof and/or deliver electrical therapy thereto. Such electrode(s) can be located at the distal portion 104 of the housing 102 and/or incorporated into the fixation mechanism 108, for example. The device 100 can also include at least one electrode (e.g., an anode and/or return electrode) that does not directly contact cardiac tissue. Such electrode(s) can be located at portions of the housing 102 that are spaced apart from cardiac tissue, such as the proximal portion 106. Optionally, a single electrode may serve as a cathode for certain operations, and may serve as an anode and/or return electrode for other operations.

In some embodiments, the device 100 is operably coupled to an external device 110 shown schematically) via bidirectional wireless communication, such as BLUETOOTH®, Wi-Fi, Medical Implant Communication Service (MICS), or other radiofrequency communication technique. The external device 110 can be a computing device or system that is located outside of the patient's body, and can be used in a healthcare setting (e.g., in a clinic, hospital or other medical facility), at the patient's home, or suitable combinations thereof. The external device 110 can be configured to control various operational parameters of the device 100, such as therapy parameters (e.g., pacing control parameters such as pacing interval), sensing parameters, power management parameters, etc. For instance, the external device 110 can transmit control signals to the device 100 to program one or more operational parameters of the device 100. Optionally, the external device 110 can display information relating to and/or received from the device 100, such as intracardiac electrogram (EGM) signals obtained by the device 100, motion sensor signals acquired by the device 100, operational parameters of the device 100, etc. In some embodiments, the external device 110 transmits information received from the device 100 to another computing device or system (e.g., a computer, laptop, workstation, mobile device, server, remote patient management system) for display, processing, and/or storage, using any suitable wired or wireless communication technique. The external device 110 can serve as a “programmer” that allows a physician, patient, or other individual to monitor and/or control the operations of the device 100.

Although FIG. 1 illustrates a single device 100, the present technology is also applicable to implantable systems including multiple devices 100 implanted at different locations in the heart H. For example, an implantable system can include a first device 100 in the RA and a second device 100 in the RV. In such embodiments, each device 100 can independently have any of the features described herein.

FIG. 2 is a perspective view of a pacing device 200 configured in accordance with embodiments of the present technology. The device 200 is configured to be implanted within a chamber of a heart of the patient to monitor activity of the heart and/or provide electrical therapy (e.g., pacing therapy) to the heart. The device 200 includes a housing 202 having a size and form factor that allows the device 200 to be entirely implanted within a single chamber of the patient's heart. In the illustrated embodiment, the housing 202 has an elongate shape (e.g., a generally cylindrical shape, a generally prismatic shape) extending between a distal end 204 and proximal end 206. The housing 202 can define a hermetically sealed internal cavity for housing the electronic components of the device 200. The housing 202 can also include an attachment mechanism 208 (e.g., at the proximal end 206) configured to temporarily engage with a delivery tool during implantation and/or extraction of the device 200.

The housing 202 can be formed partially or entirely from a conductive material, such as titanium or titanium alloy, stainless steel, MP35N (a non-magnetic nickel-cobalt-chromium-molybdenum alloy), a platinum alloy, or other biocompatible metal or metal alloy, or other suitable conductive material. Alternatively or in combination, the housing 202 can be formed partially or entirely from a nonconductive (e.g., insulative) material, such as ceramic, glass, sapphire, silicone, polyurethane, epoxy, acetyl co-polymer plastics, polyether ether ketone (PEEK), a liquid crystal polymer, other biocompatible polymer, or other suitable nonconductive material.

The device 200 can include a plurality of electrodes 210a-210c configured to sense electrical activity of the heart and/or deliver electrical stimulation to the heart. In the illustrated embodiment, for example, the device 200 includes a first electrode 210a and a second electrode 210b at or proximate to the distal end 204 of the housing 202, and a third electrode 210c on the housing 202. The first and second electrodes 210a, 210b can be configured as cathode electrodes that directly contact cardiac tissue, e.g., a distal end of the first electrode 210a can be configured to rest within a ventricular myocardium of the patient, and the second electrode 210b can be configured to contact an atrial endocardium of the patient. The third electrode 210c can be configured as an anode and/or return electrode that does not directly contact cardiac tissue.

As shown in FIG. 2, the first electrode 210a can be an elongate structure that extends from the distal end 204 of the housing 202 to penetrate through the wall tissue of a first heart chamber (e.g., the chamber in which the device 200 is implanted) into wall tissue of a second, different heart chamber. For example, in some embodiments, the device 200 is implanted in the RA with the distal end 204 oriented toward the LV (e.g., similar to the arrangement of the device 100 in FIG. 1), and the first electrode 210a extends through the wall tissue of the RA and into the wall tissue of the LV. In the illustrated embodiment, the first electrode 210a is configured as a coil (e.g., a helical and/or spiral coil), while in other embodiments, the first electrode 210a can have a different form factor (e.g., an elongate dart, barb, tine, or other tissue penetrating element). The first electrode 210a can include a proximal end that is coupled to the distal end 204 of the housing 202, and a free distal end that is not attached to the housing 202. The distal end of the first electrode 210a can have a conical, hemi-spherical, or slanted edge distal tip with a narrow tip diameter (e.g., less than 1 mm) for penetrating into and through tissue layers. In some embodiments, the distal end of the first electrode 210a can have a sharpened or angular tip, and/or sharpened or beveled edges, but the degree of sharpness can be constrained to avoid a cutting action that could lead to lateral displacement of the distal end of the first electrode 210a and undesired tissue trauma.

The second electrode 210b can be a structure that extends from the distal end 204 of the housing 202 to contact the wall tissue of the first heart chamber without penetrating the wall tissue. The second electrode 210b can be located proximal to the first electrode 210a. The second electrode 210b can be configured as a coil (e.g., a partial helical and/or spiral coil that does not form a full turn), loop, button, pad, or any other suitable form factor. The second electrode 210b can include a proximal end that is coupled to the distal end 204 of the housing 202, and a distal end that may or may not be coupled to the housing 202. In some embodiments, the second electrode 210b is configured to flexibly maintain contact with wall tissue of the heart chamber in which the device 200 is implanted, (e.g., the RA endocardium), despite variations in the tissue surface and/or in the distance between the distal end 204 of the housing 202 and the tissue surface, which may occur as the wall tissue moves during the cardiac cycle. Accordingly, the second electrode 210b can be flexible and/or have spring-like properties, e.g., the second electrode 210b can have a spring bias that urges at least a portion of the second electrode 210b away from the distal end 204 of the housing 202 and toward the wall tissue of the heart chamber to maintain consistent contact.

The first and second electrodes 210a, 210b can each be formed of an electrically conductive material, such as titanium, platinum, iridium, tantalum, or alloys thereof. The first electrode 210a can include one or more insulative coatings (e.g., parylene, polyurethane, silicone, epoxy) that reduce the electrically conductive surface area of the first electrode 210a to define a first electrically active region 212 (e.g., at or near the distal end of the first electrode 210a). The second electrode 210b can include one or more insulative coatings (e.g., parylene, polyurethane, silicone, epoxy) that reduce the electrically conductive surface area of the second electrode 210b to define a second electrically active region 214 (e.g., at an intermediate region between the proximal and distal ends of the second electrode 210b). This approach can increase the electrical impedance of the first and second electrodes 210a, 210b, and thereby reduce the current delivered during a pacing pulse, which can conserve the power used by the device 200. In some embodiments, the first and second electrodes 210a, 210b include an electrically conductive material coating (e.g., TiN) on the first and second electrically active regions 212, 214, respectively, to define the active regions. The first and second electrodes 210a, 210b can be made of the same materials, or can be made of different materials.

All, substantially all, or a portion of the housing 202 can serve as a third electrode 210c (e.g., an anode and/or return electrode) during pacing and/or sensing. In some embodiments, the third electrode 210c partially or fully circumscribes a portion of the housing 202 at or near the proximal end 206. Although FIG. 2 illustrates the third electrode 210c as a singular band, in other embodiments, the third electrode 210c can include multiple segments spaced a distance apart along a longitudinal axis 216 of the housing 202 and/or around a perimeter of the housing 202. Additionally, the third electrode 210c can also be located at other positions along the housing 202, e.g., located at or near the distal end 204 or at other positions along the longitudinal axis 216.

In embodiments where the housing 202 is formed from a conductive material, one or more portions of the housing 202 can be electrically insulated by a nonconductive material, such as a coating of parylene, polyurethane, silicone, epoxy or other biocompatible polymer, or other suitable material. For the portions of the housing 202 without the nonconductive material, one or more discrete areas of the housing 202 with conductive material can be exposed to define the third electrode 210c. In embodiments where the housing 202 is formed from a nonconductive material, a conductive material can be applied to one or more discrete areas of the housing 202 to form the third electrode 210c. Optionally, the third electrode 210c can be a discrete component (e.g., a ring electrode) that is coupled to the housing 202.

The electrodes 210a-210c can be used to sense electrical activity of one or more heart chambers and/or to deliver electrical stimulation to one or more heart chambers. For example, the first electrode 210a can be paired with the second electrode 210b or the third electrode 210c to for sensing ventricular signals and delivering ventricular pacing pulses. As another example, the second electrode 210b can be paired with the first electrode 210a or the third electrode 210c for sensing atrial signals and delivering pacing pulses to the atrial myocardium. In a further example, the third electrode 210c can be paired at different times with both the first electrode 210a and the second electrode 210b for either ventricular or atrial functionality, respectively. As yet another example, the first electrode 210a and the second electrode 210b can be paired with each other with different polarities for atrial and ventricular functionality.

In some embodiments, the second electrode 210b is configured as an atrial cathode electrode for delivering pacing pulses to the atrial tissue at a target implant region in combination with the third electrode 210c. The second electrode 210b and the third electrode 210c can also be used to sense atrial P-waves for use in controlling atrial pacing pulses (e.g., delivered in the absence of a sensed P-wave) and for controlling atrial-synchronized ventricular pacing pulses delivered using the first electrode 210a as a cathode and the third electrode 210c as the return anode. The configuration of the electrodes 210a-210c illustrated in FIG. 2 allows the device 200 to sense cardiac signals from and/or deliver cardiac pacing to one or more chambers of the heart. For example, the present technology can facilitate the delivery of A-V synchronous pacing using a single device 200 implanted within a single heart chamber (e.g., the RA).

The device 200 can include a fixation mechanism 218 configured to fix the device 200 to cardiac tissue at a target implant region (e.g., the triangle of Koch). In the illustrated embodiment, the first electrode 210a and/or second electrode 210b at the distal end 204 of the housing 202 can serve as the fixation mechanism 218. In other embodiments, the fixation mechanism 218 can be a different component than the first electrode 210a and/or the second electrode 210b, such one or more separate barbs, tines, coils, darts, etc.

FIG. 3 is a side view of another pacing device 300 configured in accordance with embodiments of the present technology. The device 300 is configured to be implanted within a chamber of a heart of a patient to monitor activity of the heart and/or to provide electrical therapy to the heart. In the embodiment shown in FIG. 3, the device 300 includes a housing 302, a plurality of fixation tines 304, a first electrode 306a, and a second electrode 306b.

The housing 302 can have a size and form factor that allows the device 300 to be entirely implanted within a chamber of a heart of a patient. For example, as shown in FIG. 3, the housing 302 has a generally cylindrical (e.g., pill-shaped or capsule-shaped), elongate form factor extending between a distal end 308 and a proximal end 310. The housing 302 contains electronic components of the device 300, and can be hermetically or near-hermetically sealed to prevent fluid ingress into the housing 302. The materials used to form the housing 302 can include any of the conductive and nonconductive materials described above with respect to FIG. 2.

The device 300 can include a fixation mechanism configured to fix the device 300 to cardiac tissue at a target implant region (e.g., the endocardial wall near the apex of the RV). In the illustrated embodiment, the device 300 includes a plurality of fixation tines 304 extending from the distal end 308 of the housing 302 and configured to engage with cardiac tissue to secure the housing 302 at a fixed position within the chamber of the heart. The fixation tines 304 can be configured to anchor the housing 302 to the cardiac tissue such that the device 300 moves along with the cardiac tissue during cardiac contractions. The device 300 can include any suitable number of fixation tines 304, such as one, two, three, four, five, or more fixation tines 304. The fixation tines 304 can be fabricated from any suitable material, such as a shape memory material (e.g., Nitinol). Alternatively or in combination, the device 300 can be fixed to cardiac tissue using other types of fixation mechanisms, such as, but not limited to, barbs, coils, darts, and the like.

Optionally, the device 300 can include an attachment mechanism configured to temporarily couple the device 300 to a delivery tool, e.g., for delivery and/or extraction of the device 300. In the illustrated embodiment, for example, the proximal end 310 includes a flange 318 that defines an opening. The flange 318 can be attached to a tether (e.g., by threading the tether through the opening) that extends through an elongate shaft (e.g., a catheter) to implant or extract the device 300.

In some embodiments, the device 300 is configured to sense electrical activity of the heart and/or deliver electrical stimulation to the heart via the first electrode 306a and second electrode 306b (collectively, “electrodes 306”). The first electrode 306a can serve as a cathode configured to electrically contact cardiac tissue and deliver pacing pulses thereto, and the second electrode 306b can serve as an anode and/or a return electrode. Optionally, the device 300 can be equipped with multiple cathode electrodes. Such multiple cathode electrodes can be configured to electrically contact and deliver pacing pulses to cardiac tissue of a single heart chamber, or cardiac tissue of multiple heart chambers. In some such embodiments, the multiple cathode electrodes are configured to electrically contact and deliver pacing pulses to cardiac tissue of different heart chambers. For example, one cathode electrode can be configured to electrically contact and deliver pacing pulses to atrial tissue, and another cathode electrode may be configured to electrically contact and deliver pacing pulses to ventricular tissue.

The electrodes 306 can be configured in many different ways. For example, one or both of the electrodes 306 can be discrete components that are mechanically coupled to the housing 302. As another example, one or both of the electrodes 306 can be defined by an outer portion of the housing 302 that is electrically conductive. The electrodes 306 can be electrically isolated from each other. In some embodiments, a portion of the housing 302 is covered by or formed from an insulative material to isolate the electrodes 306 from each other and/or to provide a desired size and shape for one or both of the electrodes 306. The electrodes 306 can be electrically coupled to at least some of the internal electronic components of the device 300 within the housing 302 (e.g., sensing circuitry, electrical stimulation circuitry, or both).

In the illustrated embodiment, the first electrode 306a is located at the distal end 308 of the housing 302. The first electrode 306a may be referred to as a tip electrode, and the fixation tines 304 can be configured to anchor the device 300 to cardiac tissue such that the first electrode 306a maintains contact with the cardiac tissue. In some examples, the housing 302 includes an end cap 312 at the distal end 308, and the end cap 312 includes a feedthrough assembly to electrically couple the first electrode 306a to the electronics within the housing 302, while electrically isolating the first electrode 306a from the remaining portions of the housing 302, e.g., including the second electrode 306b and/or other conductive portions of the housing 302

The second electrode 306b can be located on the housing 302 away from (e.g., proximal to) the first electrode 306a. As shown in FIG. 3, the housing 302 includes a first portion 314 and a second portion 316, with the first portion 314 being located proximal to the end cap 312, and the second portion 316 being located proximal to the first portion 314. The second portion 316 can optionally define at least part of a power source case that houses a power source (e.g., a battery) of the pacing device 300. In some embodiments, the second electrode 306b is located on the second portion 316, while in other embodiments, the second electrode 306b is located on the first portion 314.

In some embodiments, the second electrode 306b is a conductive portion of the housing 302 (e.g., an annular portion of the housing 302 that is made partially or entirely from a conductive material). Additionally or alternatively, the second electrode 306b can be a conductive material that is coated onto the material of the housing 302, or a discrete component (e.g., a ring electrode) that is coupled to the housing 302. The remaining portions of the housing 302 can include or be coated with an insulative material so that the second electrode 306b is electrically isolated from the rest of the housing 302 and/or from the first electrodes 306a.

FIG. 4 is a schematic block diagram illustrating electronic components of a pacing device 400 configured in accordance with embodiments of the present technology. Any of the electronic components shown in FIG. 4 can be incorporated into any of the embodiments of implantable devices described herein, such as the device 100 of FIG. 1, the device 200 of FIG. 2, or the device 300 of FIG. 3.

As shown in FIG. 4, the device 400 includes a plurality of electrodes 402a-402c that are electrically coupled to components within a housing 404 of the device 400. Although the device 400 is illustrated and described herein as having three electrodes 402a-402c (e.g., similar to the device 200 of FIG. 2), in other embodiments, the device 400 can be modified to include a different number of electrodes, such as two electrodes (e.g., similar to the device 300 of FIG. 3) or any other suitable number of electrodes.

At least some of the electrodes 402a-402c can be configured to contact tissue of one or more heart chambers, as described elsewhere herein. For example, as discussed above with respect to FIG. 2, the first electrode 402a can be configured to electrically contact and deliver electrical signals to tissue of a first heart chamber (e.g., ventricular tissue), and the second electrode 402b can be configured to electrically contact and deliver electrical signals to tissue of a second, different heart chamber (e.g., atrial tissue). The third electrode 402c can be an anode and/or return electrode that does not electrically contact heart tissue. Optionally, either the first electrode 402a or the second electrode 402b can be omitted, or the device 400 can include additional electrodes that electrically contact and deliver electrical signals to tissue of a heart chamber (e.g., the first heart chamber, the second heart chamber, or another heart chamber).

The device 400 includes a plurality of electronic components within the housing 404, such as switch circuitry 406, sensing circuitry 408, therapy generation circuitry 410, one or more sensors 412, processing circuitry 414, communication circuitry 416, memory 418, and/or a power source 420. The various circuitry can be or include programmable or fixed function circuitry configured to perform the operations described herein. One or more of the components of the device 400 shown in FIG. 4 can be part of an electronics assembly. For example, one or more of the switch circuitry 406, sensing circuitry 408, therapy generation circuitry 410, sensor(s) 412, processing circuitry 414, communication circuitry 416, and/or memory 418 can be mounted on a circuit board of an electronics assembly of the device 400.

The switch circuitry 406 can include one or more switches (e.g., a switch matrix, switch arrays, or other collection of switches), multiplexers, transistors, and/or other electrical circuitry. The switch circuitry 406 can selectively couple one or more of the electrodes 402a-402c to other components of the device 400 (e.g., the sensing circuitry 408 and/or the therapy generation circuitry 410). The subset of the electrodes 402a-402c to be used can depend on the particular operation of the device 400 that is being performed, such as whether the device 400 is sensing or delivering therapy, the locations of the heart being monitored or treated, etc. In some embodiments, the processing circuitry 414 determines which subset of the electrodes 402a-402c should be used for a particular operation, and controls the switch circuitry 406 to selectively couple those electrodes to the appropriate components of the device 400.

The sensing circuitry 408 can monitor signals from at least one of electrodes 402a-402c to monitor electrical activity of the heart, impedance, and/or other electrical phenomena. Sensing can be performed to determine heart rates and/or heart rate variability, and/or to detect ventricular dyssynchrony, arrhythmias (e.g., tachyarrhythmias), and/or other electrical signals. The sensing circuitry 408 can include filters, amplifiers, analog-to-digital converters, and/or other circuitry configured to sense cardiac electrical signals via one or more of the electrodes 402a-402c.

In some embodiments, the switch circuitry 406 as controlled by the processing circuitry 414 selectively couples the sensing circuitry 408 to selected combinations of the electrodes 402a-402c, e.g., to selectively sense the electrical activity of one or more chambers of the heart. For example, the switch circuitry 406 can couple each of the first electrode 402a and the second electrode 402b (in combination with the third electrode 402c) to respective sensing channels provided by the sensing circuitry 408 to sense electrical signals from the cardiac tissues in electrical contact with the first electrode 402a (e.g., ventricular tissue) and the second electrodes 402b (e.g., atrial tissue), respectively. In some embodiments, the sensing circuitry 408 is configured to detect events, (e.g., depolarizations) within the cardiac electrical signals, and to provide indications thereof to the processing circuitry 414. In this manner, the processing circuitry 414 can determine the timing of atrial and/or ventricular depolarizations, and can control the delivery of cardiac pacing (e.g., AV synchronized cardiac pacing) based thereon.

The therapy generation circuitry 410 can generate electrical stimulation signals, such as cardiac pacing pulses. The therapy generation circuitry 410 can be electrically coupled to one or more of the electrodes 402a-402c to deliver pulses to a portion of cardiac muscle within the heart via one or more of the electrodes 402a-402c. In some embodiments, the therapy generation circuitry 410 delivers pacing stimulation in the form of electrical pulses. The therapy generation circuitry 410 can include charging circuitry, and one or more charge storage devices (e.g., capacitors). Optionally, the therapy generation circuitry 410 can include switches and/or other circuitry to control when the charge storage devices are discharged to the electrodes 402a-402c.

The switch circuitry 406 as controlled by the processing circuitry 414 can direct electrical stimulation signals from the therapy generation circuitry 410 to a selected combination of the electrodes 402a-402c having selected polarities, e.g., to selectively deliver pacing pulses to the RA, RV, LV, and/or the interventricular septum of the heart. For example, in order to pace one or both of the ventricles, the switch circuitry 406 can electrically couple the first electrode 402a (e.g., which contacts wall tissue of a ventricle or the intraventricular septum) to the therapy generation circuitry 410 as a cathode, and to one or both of the second electrode 402b or the third electrode 402c to the therapy generation circuitry 410 as an anode. As another example, in order to pace the RA, the switch circuitry 406 can couple the second electrode 402b (e.g., which contacts the RA endocardium) to the therapy generation circuitry 410 as a cathode, and to one or both of the first electrode 402a or the third electrode 402c to the therapy generation circuitry 410 as an anode.

The processing circuitry 414 can include one or more processors, such as a microprocessor, a controller, a digital signal processor (DSP), an application specific integrated circuit (ASIC), a field-programmable gate array (FPGA), or equivalent discrete or analog logic circuitry. In some embodiments, the processing circuitry 414 can include multiple components, such as any combination of one or more microprocessors, controllers, DSPs, ASICs, and/or FPGAs, as well as other discrete or integrated logic circuitry. The functions attributed to the processing circuitry 414 herein may be embodied as software, firmware, hardware, or any combination thereof.

The processing circuitry 414 can control the therapy generation circuitry 410 to deliver stimulation therapy to a patient's heart according to therapy parameters, which can be stored in the memory 418. For example, the processing circuitry 414 can control the therapy generation circuitry 410 to deliver electrical pulses with the amplitudes, pulse widths, rates, frequencies, and/or electrode polarities specified by the therapy parameters. In this manner, the therapy generation circuitry 410 can deliver pacing pulses to the heart via one or more of the electrodes 402a-402c. The device 400 can use any combination of the electrodes 402a-402c to deliver therapy and/or detect electrical signals from the patient.

The memory 418 (e.g., a data storage device or other non-transitory medium) can store computer-readable instructions that, when executed by the processing circuitry 414, cause the device 400 to perform the various operations described herein. The memory 418 can include any volatile, non-volatile, magnetic, optical, or electrical media, such as a random-access memory (RAM), read only memory (ROM), non-volatile RAM (NVRAM), electrically-erasable programmable ROM (EEPROM), flash memory, or any other digital or analog media.

The sensor(s) 412 can include one or more sensing elements that transduce patient physiological activity to an electrical signal to sense values of a respective patient parameter. Sensor(s) 412 can include one or more motion sensors, optical sensors, chemical sensors, temperature sensors, pressure sensors, and/or any other types of sensors. The sensor(s) 412 can output patient parameter values to the processing circuitry 414 that can be used as feedback to control sensing and/or delivery of therapy by the device 400.

For example, the sensor(s) 412 can include at least one motion sensor, such as one or more inertial measurement units (IMUs), accelerometers, gyroscopes, electrical or magnetic field sensors, and/or other devices capable of detecting motion and/or the position of the device 400. The motion of the device 400 detected by the motion sensor may be indicative of cardiac events (e.g., paced activation of the ventricles), blood flow through the heart, patient posture, patient activity, and/or noise. The processing circuitry 414 can control and/or monitor the motion data produced by the motion sensor to identify one or more features of the cardiac contraction within the signal (e.g., on a beat-by-beat basis or otherwise) to facilitate delivery of therapy (e.g., delivery of ventricular pacing pulses in an atrial-synchronized manner). Optionally, the processing circuitry 414 can use the motion data to detect a current activity level of the patient, which can be used for rate-responsive pacing of the patient's heart.

The communication circuitry 416 is configured to allow the device 400 to wirelessly communicate with another device, such as a device external to the patient's body (e.g., the external device 110 of FIG. 1) and/or another device under the control of the processing circuitry 414. For instance, the processing circuitry 414 can receive updates to operational parameters from the other device, and/or can provide collected data, (e.g., sensed heart activity and/or other patient parameters) to the other device via the communication circuitry 416. The communication circuitry 416 can use radiofrequency (RF) communication techniques (e.g., via an antenna) and/or any other suitable communication modality.

The power source 420 delivers operating power to various components of the device 400. The power source 420 can include one or more batteries, each of which can independently be rechargeable or non-rechargeable. Recharging of the power source 420 can be accomplished through proximal inductive interaction between an external charger and an inductive charging coil within the device 400. Alternatively or in combination, recharging of the power source 420 can be accomplished using an energy harvesting mechanism 422 of the device 400. Additional details of energy harvesting mechanisms and associated methods are provided in Section II below.

The components of the device 400 illustrated in FIG. 4 can be modified in many different ways. For example, any of the components shown in FIG. 4 can be combined with each other, e.g., the switch circuitry 406 can be incorporated into the sensing circuitry 408 and/or the therapy generation circuitry 410. Any of the components shown in FIG. 4 can be divided into smaller subcomponents. Some of the components in FIG. 4 are optional and may be omitted (e.g., the switch circuitry 406 and/or sensor(s) 412). The device 400 can also include additional components not shown in FIG. 4. For example, the device 400 can include power management circuitry coupled to the power source 420 to allow the processing circuitry 414 to monitor the status of the power source 420 (e.g., charge level, charging rate, net power into and/or out of the power source 420, remaining battery life).

The components of the device 400 shown in FIG. 4 represent functionality that can be included in any of the devices of the present technology. The components illustrated in FIG. 4 can include any discrete and/or integrated electronic circuit components that implement analog and/or digital circuits capable of producing the functions attributed to the components herein. For example, the components can include analog circuits, such as amplification circuits, filtering circuits, and/or other signal conditioning circuits. The components can also include digital circuits, such as combinational or sequential logic circuits, memory devices, and the like. The functions attributed to the components of FIG. 4 may be embodied as one or more processors, hardware, firmware, software, or any combination thereof. The depiction of different features as separate blocks in FIG. 4 is intended to highlight different functional aspects, and does not necessarily imply that such components must be realized by separate hardware or software components. Rather, functionality associated with one or more components may be performed by separate hardware or software components, or integrated within common or separate hardware or software components. For example, although illustrated as separate functional components in FIG. 4, some or all of the functionality attributed to the switch circuitry 406, sensing circuitry 408, therapy generation circuitry 410, sensor(s) 412, and/or communication circuitry 416 can alternatively or additionally be implemented by the processing circuitry 414, or vice-versa.

### II. Methods and Devices for Stimulation Control for Energy Harvesting

In some embodiments, the present technology provides implantable devices that include an energy harvesting mechanism (also known as an “energy harvester” or “harvester”). The power capacity of a power source of an implantable device may be limited due to size constraints, such as if the device is implanted within a small space within the patient's body (e.g., within a single heart chamber) and/or to avoid the device interfering with normal physiological function, as well as safety considerations. To prolong the usable life of such implantable devices, an energy harvesting mechanism can be used to generate energy in situ to recharge the power source.

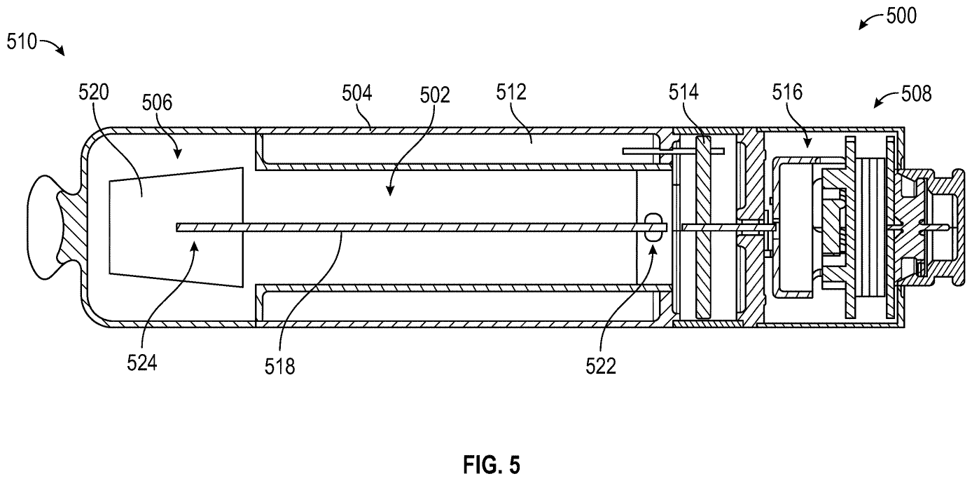

FIG. 5 is a side cross-sectional view of a device 500 including an energy harvesting mechanism 502, in accordance with embodiments of the present technology. The device 500 can be an implantable device, such as a pacing device configured to monitor activity of a patient's heart and provide electrical stimulation to the heart. In such embodiments, the device 500 can include any of the features of the devices described above in connection with FIGS. 1-4 (e.g., electrodes, fixation mechanism, circuitry and/or other electronic components). In other embodiments, however, the device 500 can be a different type of implantable medical device.

The device 500 includes a housing 504 having an elongate shape extending between distal end 508 and a proximal end 510. The housing 504 defines an interior cavity 506 containing the energy harvesting mechanism 502 and other components of the device 500, such as a power source 512, power conditioning circuitry 514, and an electronics assembly 516. When the device 500 is implanted in a patient's body, the energy harvesting mechanism 502 generates energy from physiological motion. For example, in some embodiments, the device 500 is configured to be implanted within a heart chamber of the patient and generates energy from cardiac motion (e.g., motion of the heart wall to which the device 500 is affixed) and/or blood flow through the heart chamber. The energy produced by the energy harvesting mechanism 502 can be used to charge the power source 512, which in turn powers the operation of the device 500.

In some embodiments, the energy harvesting mechanism 502 includes a piezoelectric element 518 that converts mechanical energy into electrical energy via the piezoelectric effect. The piezoelectric element 518 can be or include a flexible elongate member (e.g., beam, plate, shaft, rod, fiber) made partially or entirely out of a piezoelectric material, such as a piezoelectric ceramic (e.g., lead zirconate titanate (PZT)), a piezoelectric polymer (e.g., polyvinylidene difluoride (PVDF)), or a piezoelectric composite (e.g., a piezoelectric ceramic embedded in a polymer matrix, such as a macro fiber composite). The piezoelectric element 518 can be in a cantilever configuration in which a first end 522 of the piezoelectric element 518 is fixed relative to the housing 504, and a second end 524 of the piezoelectric element 518 opposite the first end 522 is movable relative to the housing 504. In the illustrated embodiment, the first end 522 of the piezoelectric element 518 is located near the distal end 508 of the housing 504, the second end 524 of the piezoelectric element 518 is located near the proximal end 510 of the housing 504, and the longitudinal axis of the piezoelectric element 518 is aligned with (e.g., parallel to) the longitudinal axis of the housing 504. In other embodiments, however, the piezoelectric element 518 can be oriented differently with respect to the housing 504. Additionally, the energy harvesting mechanism 502 can optionally include multiple piezoelectric elements 518.

In some embodiments, the second end 524 of the piezoelectric element 518 is coupled to a harvester mass 520 (also known as a “proof mass” or “inertial mass”). Due to the inertia of the harvester mass 520, when the device 500 is subjected to external forces from physiological motion, the harvester mass 520 can cause displacement of the second end 524 of the piezoelectric element 518 relative to the housing 504 and the fixed first end 522 of the piezoelectric element 518, and thus cause elastic deformation of the piezoelectric element 518. For instance, the piezoelectric element 518 can be deformed from a resting, straightened configuration (shown in FIG. 5) to a bent configuration (e.g., an upwardly bent configuration or a downwardly bent configuration). The resulting mechanical strain in the piezoelectric element 518 can produce an electrical current that can be used to charge the power source 512.

The power source 512 can include one or more rechargeable batteries that are electrically coupled to the energy harvesting mechanism 502 to store the energy produced by the energy harvesting mechanism 502. In the illustrated embodiment, the power source 512 is configured as a tubular structure that surrounds at least a portion of the energy harvesting mechanism 502 (e.g., an intermediate portion of the piezoelectric element 518 between the distal end 508 and the proximal end 510). This configuration can be advantageous for reducing the overall size of the device 500 while maintaining sufficient space within the interior cavity 506 to allow for movement of the harvester mass 520 and piezoelectric element 518. In other embodiments, however, the power source 512 can have a different shape and/or can be located at a different portion within the housing 504.

In some embodiments, the device 500 includes power conditioning circuitry 514 electrically coupled to and interposed between the energy harvesting mechanism 502 and the power source 512. The power conditioning circuitry 514 can be configured to perform operations such as rectification, filtering, voltage regulation, etc., of the electrical signal produced by the energy harvesting mechanism 502, before transmission to the power source 512.

The power source 512 is electrically coupled to the electronics assembly 516 to power the operation thereof. The electronics assembly 516 can include the electronic components of the device 500, such as any of the components described above with respect to FIG. 4 (e.g., switch circuitry 406, sensing circuitry 408, therapy generation circuitry 410, sensors 412 processing circuitry 414, communication circuitry 416, and/or memory 418). Optionally, the electronics assembly 516 can include components (e.g., processing circuitry 414 and/or other circuitry) that perform power management functions, such as monitoring the status of the power source 512 (e.g., the charge level of the power source 512; whether the charge level is increasing, decreasing, or constant; the net current and/or power into the power source 512) and/or monitoring the power output of the energy harvesting mechanism 502 (e.g., amount of current and/or power produced by the energy harvesting mechanism 502), power consumption of the electronics assembly 516, etc.

In some embodiments, the power output of a piezoelectric element of an energy harvesting mechanism (e.g., the piezoelectric element 518 of the energy harvesting mechanism 502 of FIG. 5) is enhanced (e.g., maximized) when one or more frequency components of the physiological motion acting upon the piezoelectric element match or are close to at least one resonant frequency of the piezoelectric element. However, it can be difficult to predict the frequencies of the physiological motion before the device is implanted into the patient. Additionally, the frequencies of the physiological motion may vary from patient to patient, and may vary even for a single patient depending on the patient's particular anatomy, physiological state, posture, and/or activity status (e.g., whether the patient is currently sleeping, resting, moving, exercising, etc.).

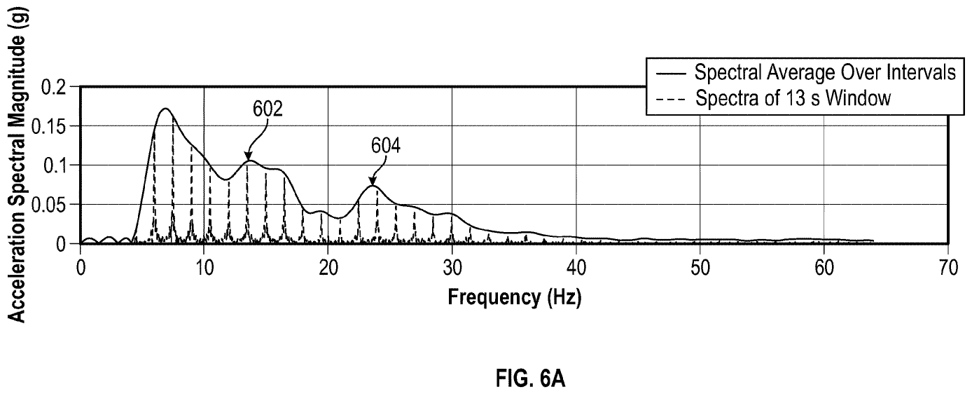

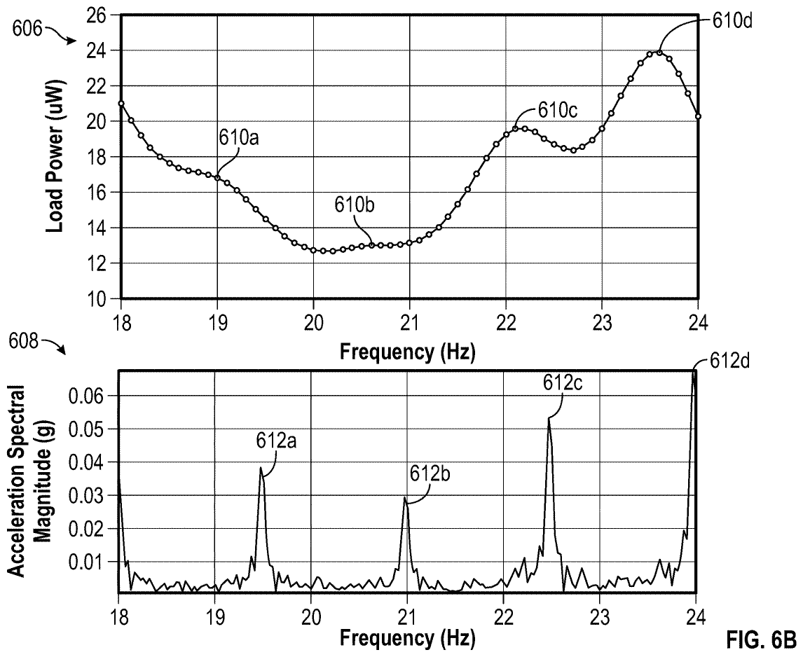

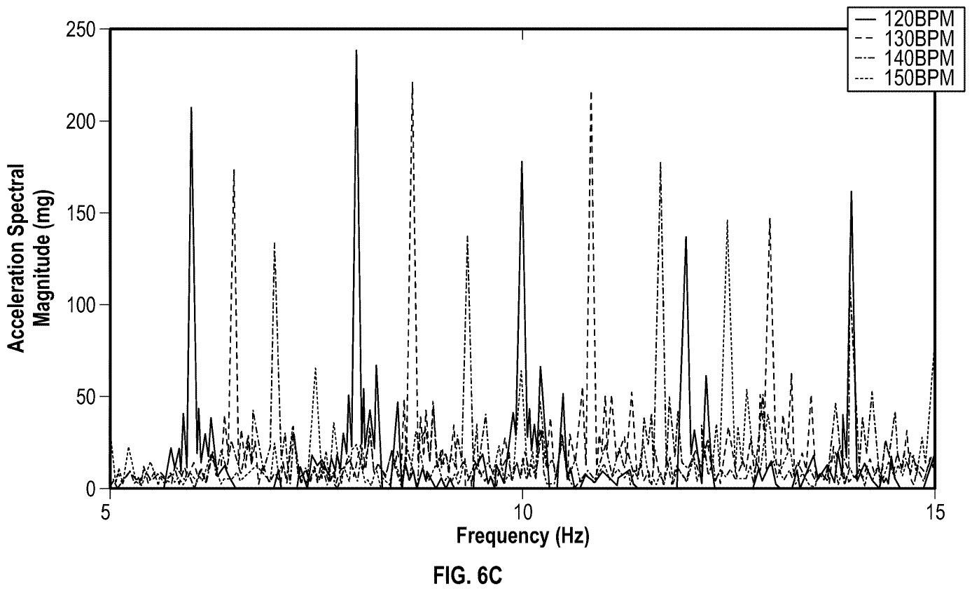

FIG. 6A is a graph illustrating an example of a frequency spectrum of cardiac motion, in accordance with embodiments of the present technology. A time domain acceleration waveform was collected using a sensor implanted in the heart of an animal model over 30 heartbeats spanning a 13 second time window. The acceleration data was processed using a high-pass filter having a 5 Hz cutoff frequency. For each beat, a fast Fourier Transform (FFT) was calculated. The 30 FFTs were averaged to determine the beat-to-beat spectral average (solid line). A FFT for the entire data set (all 30 beats) was also calculated (broken line). As shown in FIG. 6A, due to the repetitive nature of cardiac motion, the frequency spectrum of the cardiac motion includes multiple spectral peaks, such as a peak 602 at approximately 13.7 Hz and a peak 604 at approximately 23.6 Hz.

FIG. 6B illustrates a comparison between the frequency characteristics of a tunable energy harvesting mechanism (graph 606, top) and the frequency spectrum of cardiac motion (graph 608, bottom), in accordance with embodiments of the present technology. The graph 606 shows an example of how the power output (e.g., load power) of an energy harvesting mechanism can vary as the resonant frequency of the harvester varies for the input spectrum in graph 608. The local maxima (e.g., peaks 610a-610d) in the power output can correlate to spectral peaks in the input acceleration spectra. In some instances, the power output of the energy harvesting mechanism can be increased and/or maximized if one or more of the resonant frequencies of the energy harvesting mechanism (e.g., corresponding to peaks 610a-610d in graph 606) match or are sufficiently close to one or more of the spectral peaks of the frequency spectrum of the cardiac motion (e.g., peaks 612a-612d in graph 608). Conversely, the power output can be diminished if the resonant frequencies of the energy harvesting mechanism are significantly different from the spectral peaks of the cardiac motion.

FIG. 6C is a graph illustrating examples of full-time frequency spectra for cardiac motion at different heart rates (120 BPM, 130 BPM, 140 BPM, and 150 BPM), in accordance with embodiments of the present technology. Cardiac acceleration data was obtained while a cardiac pacing signal was applied using an implanted device. The cardiac pacing rate was increased in 10 BPM increments. A FFT was used to generate the frequency spectra as described above. The spectral frequency content increased as the pacing rate increased, as shown by the spectral peaks shifting to the right toward higher frequencies with higher pacing rates. This phenomenon can be used to increase the power output of an energy harvesting mechanism, as described in detail below.

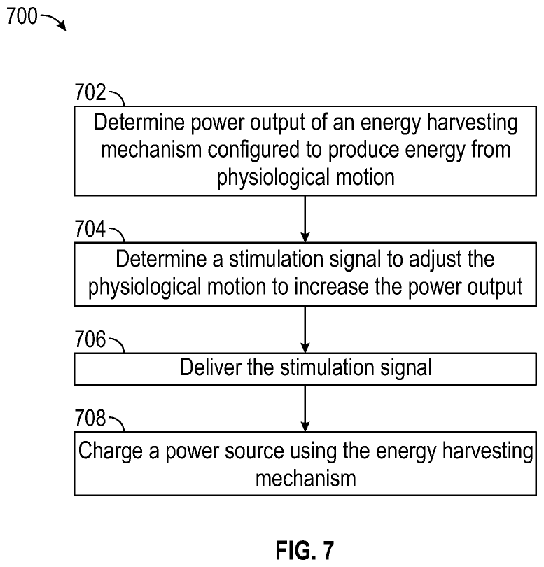

FIG. 7 is a flow diagram illustrating a method 700 for powering an implantable device, in accordance with embodiments of the present technology. The method 700 can be performed using any of the systems and devices described herein, such as any of the devices of FIGS. 1-5. In some embodiments, some or all of the processes of the method 700 are implemented as computer-readable instructions (e.g., program code) that are configured to be executed by one or more processors (e.g., processing circuitry 414 of the device 400 of FIG. 4).

The method 700 can begin at block 702 with determining a power output of an energy harvesting mechanism configured to produce energy from physiological motion. In some embodiments, the energy harvesting mechanism is a component of an implantable device, such as a device configured to be implanted in a patient's heart to deliver electrical stimulation (e.g., pacing signals) thereto, as previously described with respect to FIGS. 1-5. The energy harvesting mechanism can be a kinetic harvester that is configured to convert mechanical energy from the physiological motion into electrical energy. For instance, the energy harvesting mechanism can include a movable piezoelectric element that generates energy from cardiac motion, as described above in connection with FIG. 5.

The power output of the energy harvesting mechanism can be determined in various ways. For example, the power output can be determined by measuring the amount of power provided by the energy harvesting mechanism to a power source (e.g., to a rechargeable battery onboard the implantable device). In some embodiments, the power output is determined in terms of the net power into the power source, which can be computed by measuring the difference between the amount of provided to the power source by the energy harvesting mechanism, and the amount of power being output by the power source (e.g., for generating electrical stimulation signals and/or powering other operations of the device). Optionally, the power output can be determined based on the net current into the power source, which can be computed by measuring the difference between the current into the power source from the energy harvesting mechanism, and the current being output by the power source (e.g., pacing current), and multiplying by the voltage of the power source. The power and/or current measurements can be obtained using any suitable technique, such as by using a Coulomb counter to integrate the current into and/or out of the power source over a certain time period.

The power output measured in block 702 can be an initial (e.g., baseline) power output of the energy harvesting mechanism. The power energy output can be measured while no stimulation signal is being applied by the implantable device, or while a stimulation signal is being applied. For example, in embodiments where the energy harvesting mechanism is used to power a cardiac pacing device, the initial power output can be determined while the pacing device is not delivering any pacing signal to the heart. Alternatively, the initial power output can be determined while the pacing device is delivering an initial pacing signal to the heart, but the initial pacing signal has not been optimized for energy harvesting. For instance, the initial pacing signal can be determined based on considerations other than energy harvesting, such as an activity level of the patient.

In some embodiments, a rate-responsive pacing function is used to determine an initial pacing rate based on an activity metric of the patient, such as an activity count indicating the number of times the signal from an activity sensor (e.g., a motion sensor) crosses a threshold during an activity count interval. The activity count can be correlated to the patient's body motion and/or metabolic demand, and can be used to determine an appropriate pacing rate for the patient's current activity level. For example, the pacing rate can be determined using a rate-responsive pacing function (also known as a sensor-indicated rate (SIR) function) that identifies the appropriate pacing rate for each of a plurality of different activity counts. The pacing rate set by the rate-responsive pacing function can be used as the initial pacing rate. Additional details of techniques for rate-responsive pacing are provided in U.S. Patent Application Publication No. 2020/0121931, which is incorporated by reference herein in its entirety.

At block 704, the method 700 can continue with determining a stimulation signal configured to adjust the physiological motion to increase the power output of the energy harvesting mechanism. The stimulation signal can be or include an electrical signal that, when applied to a target region of the patient's body, causes a change in the physiological motion that drives the energy harvesting mechanism. In some embodiments, the process of block 704 includes determining one or more parameters of the stimulation signal (e.g., rate, frequency, amplitude, waveform, pulse width, duty cycle) such that the resulting physiological motion includes at least one frequency component that matches, overlaps, or is otherwise close to a resonant frequency of the energy harvesting mechanism. For instance, the resulting physiological motion can include at least one spectral peak having a peak frequency that is within 0 Hz, 0.1 Hz, 0.25 Hz, 0.5 Hz, 1 Hz, 2 Hz, 5 Hz, 10 Hz, or 20 Hz of a resonant frequency of the energy harvesting mechanism.

In some embodiments, the stimulation signal is a pacing signal that alters the cardiac rhythm of the patient's heart, and the process of block 704 includes determining one or more pacing parameters of the pacing signal, such as the rate, frequency, amplitude, waveform, pulse width, and/or duty cycle of the pacing signal. For example, the pacing rate of the pacing signals delivered to the heart can change the patient's heart rate in a manner that affects the frequency spectrum of the motion of the heart to improve the efficiency of the energy harvesting mechanism. The selected pacing rate can be a rate that causes at least one spectral peak of the frequency spectrum to match, overlap, or move closer to at least one resonant frequency of the energy harvesting mechanism. For example, the energy harvesting mechanism can have a resonant frequency within a range from 1 Hz to 50 Hz, 1 Hz to 30 Hz, 1 Hz to 20 Hz, 1 Hz to 10 Hz, 5 Hz to 10 Hz, 5 Hz to 15 Hz, 10 Hz to 20 Hz, 10 Hz to 15 Hz, 10 Hz to 30 Hz, 15 Hz to 20 Hz, 15 Hz to 25 Hz, 20 Hz to 30 Hz, 20 Hz to 25 Hz, or 25 Hz to 30 Hz. The pacing rate can shift a spectral peak of the cardiac motion to be within 0 Hz, 0.1 Hz, 0.25 Hz, 0.5 Hz, 1 Hz, 2 Hz, 5 Hz, 10 Hz, or 20 Hz of the resonant frequency. The peak frequency of the shifted spectral peak can be within a range from 1 Hz to 50 Hz, 1 Hz to 30 Hz, 1 Hz to 20 Hz, 1 Hz to 10 Hz, 5 Hz to 10 Hz, 5 Hz to 15 Hz, 10 Hz to 20 Hz, 10 Hz to 15 Hz, 10 Hz to 30 Hz, 15 Hz to 20 Hz, 15 Hz to 25 Hz, 20 Hz to 30 Hz, 20 Hz to 25 Hz, or 25 Hz to 30 Hz.

In some embodiments, increases in the pacing rate cause an increase in the peak frequency of at least one spectral peak of the cardiac motion, while decreases in the pacing rate cause a decrease in the peak frequency of at least one spectral peak of the cardiac motion. For example, the pacing rate can be increased by at least 1 BPM, 2 BPM, 5 BPM, 10 BPM, 15 BPM, or 20 BPM, which can cause the peak frequency of at least one spectral peak to increase by at least 0.1 Hz, 0.25 Hz, 0.5 Hz, 1 Hz, 2 Hz, 5 Hz, or 10 Hz. Conversely, the pacing rate can be decreased by at least 1 BPM, 2 BPM, 5 BPM, 10 BPM, 15 BPM, or 20 BPM, which can cause the peak frequency of at least one spectral peak to decrease by at least 0.1 Hz, 0.25 Hz, 0.5 Hz, 1 Hz, 2 Hz, 5 Hz, or 10 Hz. The amount of change in the spectral peak can depend on the base pacing rate, such as the fundamental frequency and/or harmonics of the base pacing rate, which can correlate the locations of the spectral peaks in the frequency spectrum. For example, a pacing rate of 60 BPM can have a fundamental frequency of 1 Hz and harmonics at 2 Hz, 3 Hz, 4 Hz, etc. (integer multiples of the fundamental frequency), while a pacing rate of 66 BPM can have a fundamental frequency of 1.1 Hz and harmonics at 2.2 Hz, 3.3 Hz, 4.4 Hz, etc.

The determined stimulation signal can adjust the physiological motion to increase the power output of the energy harvesting mechanism relative to the initial power output measured at block 702. For instance, the power output can be increased by at least 10%, 20%, 30%, 40%, 50%, 60%, 70%, 80%, 90%, 100%, 200%, 300%, 400%, or 500% relative to the initial power output. As described above, the power output of the energy harvesting mechanism can be measured in terms of the net power to the power source. In some embodiments, the net power to the power source is increased by at least 1 μW, 2 μW, 3 μW, 4 μW, 5 μW, 10 μW, 15 μW, or 20 μW relative to the initial net power measured at block 702.

In some embodiments, the determined stimulation signal adjusts the physiological motion in a manner that does not cause harm and/or discomfort to the patient, and/or without substantially affecting the therapeutic effect of the stimulation signal. For instance, in the context of cardiac pacing, the stimulation signal can be a pacing signal having a pacing rate (and/or other pacing parameters) that maintains an appropriate cardiac rhythm for the patient, such as a cardiac rhythm suitable for meeting the patient's current metabolic demands and/or for treating a cardiac condition of the patient (e.g., an abnormal cardiac rhythm such as bradycardia). The pacing rate can be within a predetermined range of a baseline pacing rate, such as a pacing rate determined based on the patient's activity level (e.g., a SIR using a rate-responsive pacing function, as discussed herein). For instance, the pacing rate can be no more than 1 BPM, 2 BPM, 3 BPM, 4 BPM, 5 BPM, 6 BPM, 7 BPM, 8 BPM, 9 BPM, or 10 BPM greater than or less than the baseline pacing rate or SIR.

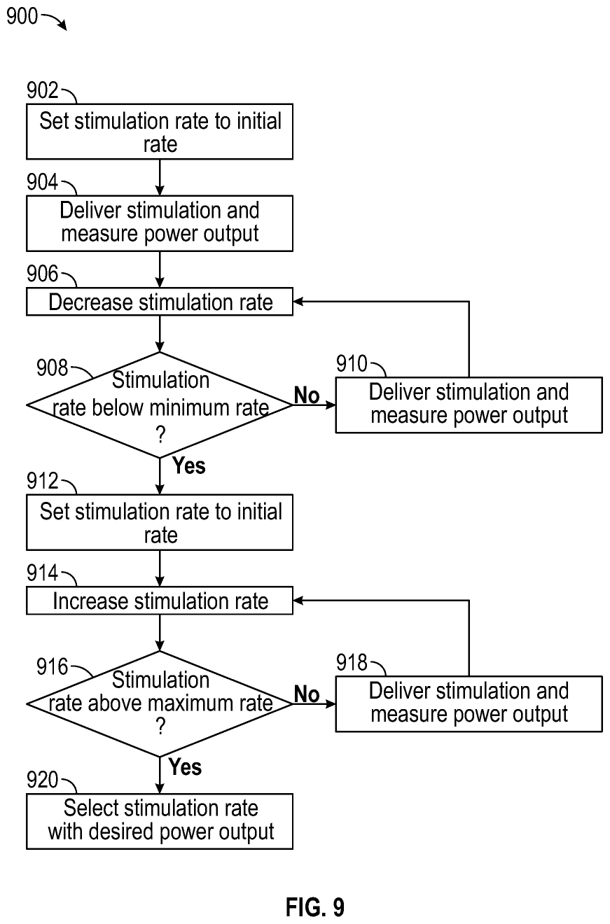

The stimulation signal that produces the increased power output can be determined in many different ways. For example, the stimulation signal can be determined on an ad hoc basis, such as by delivering a plurality of different stimulation signals to the patient, measuring the power output resulting from each stimulation signal, and selecting the stimulation signal that produces the highest power output (e.g., highest net power to the power source). The stimulation signals can differ from each other with respect to one or more stimulation parameters, such as the rate, frequency, amplitude, waveform, pulse width, duty cycle, etc. For instance, in the context of cardiac pacing, the process of block 704 can include applying pacing signals at a plurality of different pacing rates, then selecting the pacing rate that produces an increased power output, as described in detail below in connection with FIG. 9. This approach may be used in situations where the relationship between the stimulation parameters and power output is unpredictable and/or inconsistent, such as whether the power output may be affected by other factors (e.g., the current posture and/or activity of the patient).

As another example, the stimulation signal can be determined at a previous time period, such as during a previous calibration routine for the implantable device. The calibration routine can be performed to determine the relationship between the power output of the energy harvesting mechanism and the stimulation parameters of the stimulation signal. For example, the calibration routine can involve delivering a plurality of different stimulation signals to the patient (e.g., stimulation signals having one or more different stimulation parameters) and measuring the power output resulting from each stimulation signal. The calibration routine can optionally include determining the resonant frequencies of the energy harvesting mechanism, as well as the frequency spectrum and/or spectral peaks of the physiological motion for a plurality of different stimulation signals. The calibration routine can be performed at any suitable time, such as immediately after the implantable device has been implanted in the patient (e.g., as part of the setup routine for the implantable device), at periodic intervals (e.g., once per day, week, month, year), when changes are made to the configuration of the implantable device, at a time determined by a healthcare professional, or suitable combinations thereof.

The results of the calibration routine can be a transfer function representing the relationship between a plurality of stimulation signals (e.g., different pacing rates) and the corresponding power output of the energy harvesting mechanism. The calibration results can be stored as a lookup table or other suitable data structure, and can be stored onboard the implantable device (e.g., in the memory 418 of FIG. 4) and/or on a separate device (e.g., the external device 110 of FIG. 1). In such embodiments, the process of block 704 can involve retrieving the appropriate stimulation signal from the lookup table or other data structure. For instance, in the context of cardiac pacing, the appropriate pacing rate can be determined by determining a baseline pacing rate for the patient (e.g., a pacing rate determined based on the patient's current activity level and/or a SIR using a rate-responsive pacing function), then using the calibration results to identify a predetermined pacing rate that is sufficiently close to the baseline pacing rate (e.g., within 1 BPM, 2 BPM, 3 BPM, 4 BPM, 5 BPM, 6 BPM, 7 BPM, 8 BPM, 9 BPM, or 10 BPM) and produces an increased power output (e.g., increased and/or highest net power to the power source). This approach may be used in situations where the relationship between the stimulation parameters and the power output is relatively predictable and/or consistent.

As a further example, in embodiments where the stimulation signal is a pacing signal, the appropriate pacing rate to increase power output can be determined based on a pacing rate set by a rate-responsive pacing function. A correspondence between the pacing rates set by a rate-responsive function and the pacing rates for increasing power output can be generated using a calibration routine, experimental data, modeling, simulations, data from other patients, literature, and/or suitable combinations thereof. The correspondence can be represented as a transfer function indicating a modification to the pacing rate set by the rate-responsive function to maintain at least one spectral peak of the cardiac motion within a predetermined range of a resonant frequency of the cardiac motion. The transfer function can be stored as a lookup table or other suitable data structure, and can be stored onboard the implantable device (e.g., in the memory 418 of FIG. 4) and/or on a separate device (e.g., the external device 110 of FIG. 1).

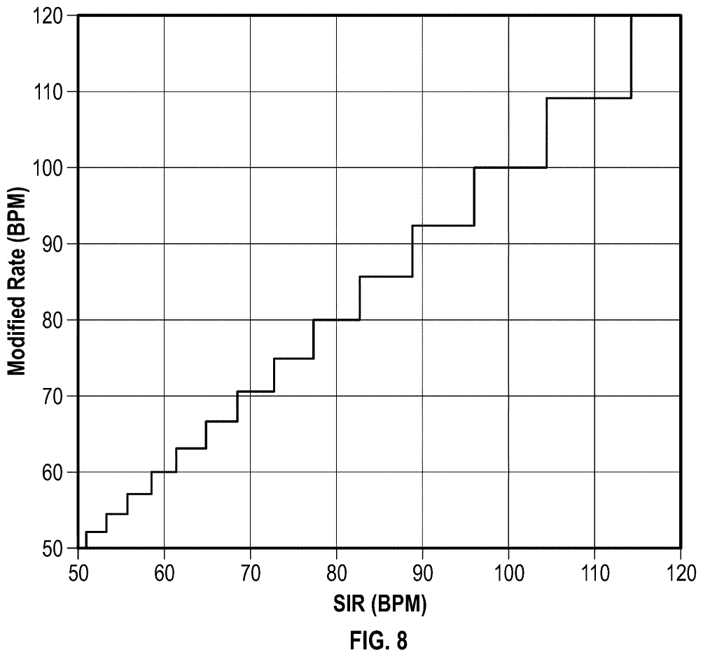

FIG. 8 is a graph illustrating an example transfer function between a pacing rate determined using a rate-responsive pacing function (“SIR”) and a modified pacing rate to enhance energy harvesting (“modified rate”), in accordance with embodiments of the present technology. In the illustrated embodiment, the resonant frequency of the energy harvesting mechanism is assumed to be 20 Hz, and the transfer function is configured to maintain a spectral peak of the cardiac motion at 20 Hz. If the Q factor of the resonance is sufficiently low, the spectral peak may not need to be exactly at the resonant frequency of the energy harvesting mechanism for efficient energy harvesting, such that greater tolerances can be allowed (e.g., within +/−0.5 Hz) and discretization can be less coarse than the example shown in FIG. 8.

Referring again to FIG. 7, to determine the appropriate pacing rate for the pacing signal to increase power output, the process of block 704 can include determining a baseline pacing rate for the patient, using the rate-responsive pacing function. The stored transfer function can then be used to look up or otherwise determine the modified pacing rate corresponding to the baseline pacing rate. The modified pacing rate may be less than, equal to, or greater than the baseline pacing rate. For example, in FIG. 8, a SIR of 80 BPM maps to a modified rate of 80 BPM (which is the same as the SIR), and a SIR of 90 BPM maps to a modified rate of 92 BPM (which is different than the SIR). The modified rate can then be used directly as the pacing rate for the pacing signal, or can be used as the starting point for an ad hoc analysis of a plurality of pacing rates, as discussed above and described in greater detail below in connection with FIG. 9.

Optionally, the stimulation signal of block 704 can be determined in other ways, such as based on experimental data, modeling, simulations, data from other patients, literature, or suitable combinations thereof. Moreover, any of the approaches described herein can be combined. In some embodiments, for example, a baseline stimulation signal is determined based on calibration results, a transfer function from a rate-responsive pacing function, and/or other sources (e.g., experimental data, modeling, simulations, data from other patients, literature). The power output of the energy harvesting mechanism can then be measured while varying one or more stimulation parameters of the baseline stimulation signal (e.g., stimulation rate, stimulation frequency, stimulation amplitude, stimulation waveform, pulse width, duty cycle), and the stimulation parameter(s) that produce an increased power output can be selected.

At block 706, the method 700 can include delivering the stimulation signal that was determined in block 704. The delivery of the stimulation signal can result in an adjustment to the physiological motion (e.g., modifying the patient's heart rate). At block 708, the method 700 can include charging a power source using the energy harvesting mechanism, driven by the adjusted physiological motion (e.g., the heart beating at the modified rate). The stimulation signal can be applied for any suitable amount of time, such as for 30 seconds, 1 minute, 5 minutes, 10 minutes, 30 minutes, 1 hour, or more; while the patient maintains a consistent activity level; while stimulation is therapeutically beneficial or needed; and so on. The power source can be charged while the stimulation signal is being delivered, after termination of the stimulation signal, or both.

In some embodiments, the stimulation signal is delivered with the stimulation parameters of block 704 (e.g., at the modified rate) until the charge level of the power source reaches a threshold value, such as full charge, or at least 50%, 60%, 70%, 75%, 80%, 90%, or 95% of full charge. Once the power source has been charged to the desired level, the stimulation signal can be continued (e.g., using the same stimulation parameters determined in block 704, such as at the modified rate, or parameters of a baseline stimulation signal), or can be terminated. Optionally, the stimulation signal can be paused or terminated, or switched from the modified rate to an un-modified rate, before the power source has been charged to the desired level, such as if the patient's condition changes in a manner such that the determined stimulation signal is no longer therapeutically appropriate. In such instances, the stimulation signal and charging can be resumed once the patient's condition returns to the previous state, or the process of block 704 can be repeated to determine a new stimulation signal that is appropriate for the patient's current condition.

For example, in the context of cardiac pacing, the stimulation signal can be a pacing signal that is delivered via one or more electrodes of a pacing device to one or more chambers of the patient's heart, as described elsewhere herein. The determined pacing signal can be delivered at a pacing rate that changes the patient's cardiac rhythm (e.g., increases or decreases the heart rate) to increase and/or maximize the amount of power produced by the energy harvesting mechanism. Once charging of the power source is complete, the pacing device can revert to delivering the pacing signal at a baseline pacing rate, such as a SIR output by a rate-responsive pacing function. In some embodiments, if the pacing device determines that the patient's activity level has changed significantly (e.g., based on motion data and/or other sensor data), such that the current pacing rate is no longer therapeutically appropriate (e.g., the current pacing rate is too fast or too slow given the patient's current activity level), the pacing device can revert to using the pacing rate set by the rate-responsive pacing function. Alternatively, the pacing device can repeat the process of block 704 to determine a new pacing rate that enhances power output of the energy harvesting mechanism, while applying a pacing signal suitable for the patient's current activity level.

The method 700 can be modified in many different ways. For example, some of the processes shown in FIG. 7 can be omitted. If it is determined that the power output of the energy harvesting mechanism is greater in the absence of any stimulation signal compared to the energy output with stimulation (e.g., the unmodified physiological motion produces the greatest energy output), the process of block 706 can be omitted so that the power source is charged without delivering stimulation.

The method 700 can also include additional processes not shown in FIG. 7. In some embodiments, for example, the energy harvesting mechanism can be adjustable to vary the resonant frequency of the energy harvesting mechanism, such as by changing the length of the piezoelectric element, location of the harvester mass along the piezoelectric element, and/or other approaches known to those of skill in the art. The method 700 can include determining an adjustment to the resonant frequency of the energy harvesting mechanism to increase the power output of the energy harvesting mechanism. The resonant frequency can be adjusted to be within 0 Hz, 0.1 Hz, 0.25 Hz, 0.5 Hz, 1 Hz, 2 Hz, 5 Hz, or 10 Hz of the peak frequency of a spectral peak of the physiological motion. In some embodiments, the method 700 includes adjusting the energy harvesting mechanism to a plurality of different resonant frequencies, measuring the power output achieved with each resonant frequency, and then selecting the resonant frequency that produces the desired (e.g., highest) power output. Accordingly, controlling the resonant frequency of the energy harvesting mechanism can be used in combination with or as an alternative to controlling the parameters of the stimulation signal to enhance energy harvesting. This approach can be advantageous in situations where it is difficult or impossible to match the resonant frequency of the energy harvesting mechanism to the spectral peaks of the physiological motion using stimulation control alone.

FIG. 9 is a flow diagram illustrating a method 900 for determining a stimulation signal to enhance power output of an energy harvesting mechanism, in accordance with embodiments of the present technology. The method 900 can be performed in combination with any of the other methods described herein. For example, the method 900 can be performed as part of the process of block 704 of the method 700 of FIG. 7, as part of a calibration routine before the method 700 is performed, etc. The method 900 can be performed using any of the systems and devices described herein, such as any of the devices of FIGS. 1-5. In some embodiments, some or all of the processes of the method 900 are implemented as computer-readable instructions (e.g., program code) that are configured to be executed by one or more processors (e.g., processing circuitry 414 of the device 400 of FIG. 4).

The method 900 can begin at block 902 with setting the stimulation rate of a stimulation signal to an initial rate. The stimulation signal can be or include an electrical signal that is configured to control and/or modify physiological motion. For example, the stimulation signal can be a pacing signal configured for pacing one or more chambers of a patient's heart, and the stimulation rate can be a pacing rate for the pacing signal. The initial rate can be a baseline rate or default rate that is determined before the current power output of the energy harvesting mechanism has been evaluated. In some embodiments, the initial rate can be determined based on the current activity level of the patient, stored results from a calibration routine, a stored transfer function based on pacing rates set by a rate-responsive function, and/or any other suitable approach. For example, in the context of cardiac pacing, the initial rate can be determined by measuring an activity metric of the patient (e.g., activity counts over a specified time interval), then using a rate-responsive pacing function to identify a pacing rate corresponding to the activity metric. The pacing rate set by the rate-responsive pacing function can be used as the initial rate for block 902; or the pacing rate set by the rate-responsive pacing function can be correlated to a modified rate (e.g., using a transfer function as shown in FIG. 8), and the modified rate can be used as the initial rate for block 902.

At block 904, the method 900 can include delivering the stimulation signal at the initial rate, and measuring the resulting power output of the energy harvesting mechanism. As described elsewhere herein, the power output can be determined in terms of the net power and/or net current to a power source that is electrically coupled to the energy harvesting mechanism, or any other suitable metric. The process of block 904 can be performed for a time period that is sufficiently long for the stimulation signal to affect the physiological motion and for the power output of the energy harvesting mechanism to be accurately measured. For instance, the process of block 904 can be performed for at least 30 seconds, 1 minute, 2 minutes, 5 minutes, or 10 minutes; and/or no more than 20 minutes, 10 minutes, 5 minutes, 2 minutes, or 1 minute.

At block 906, the method 900 can include decreasing the stimulation rate. The stimulation rate can be decreased by a predetermined (e.g., fixed) amount. For instance, in embodiments where the stimulation rate is a pacing rate, the pacing rate can be decreased by 1 BPM, 2 BPM, 3 BPM, 4 BPM, 5 BPM, 10 BPM, or 20 BPM. The magnitude of the decrease can be sufficiently large to affect the physiological motion (e.g., by shifting one or more spectral peaks of the motion by at least 0.1 Hz, 0.25 Hz, 0.5 Hz, 1 Hz, 2 Hz, 5 Hz, or 10 Hz), but not so large as to be noticeable by the patient and/or cause detrimental effects (e.g., harm and/or discomfort).

At block 908, the method 900 can include determining whether the decreased stimulation rate is below a minimum rate. The minimum rate can be a predetermined value based on considerations of patient safety (e.g., decreasing the rate below the minimum rate may cause harm and/or discomfort), therapeutic efficacy (e.g., decreasing the rate below the minimum rate may result in loss of therapeutic benefit and/or otherwise be therapeutically inappropriate), device limitations, etc. The appropriate minimum rate can be a patient-specific rate based on the particular characteristics of the patient, or can be a generic rate that is applicable to broader category of patients (e.g., patients of a particular age, weight, etc.) or to all patients. In the context of pacing rate, the minimum rate can be within 1 BPM, 2 BPM, 3 BPM, 4 BPM, 5 BPM, 10 BPM, or 20 BPM of the initial pacing rate (e.g., a SIR output by a rate-responsive pacing function).

If the decreased stimulation rate is not less than the minimum rate, the method 900 can continue to block 910 with delivering stimulation at the decreased stimulation rate and measuring the resulting power output of the energy harvesting mechanism, e.g., as described above with respect to block 904. The processes of blocks 906, 908, and 910 can be repeated to incrementally decrease the stimulation rate and determine the resulting power output, until the minimum rate is reached. The stimulation rate can be decreased by the same amount for each iteration, or can be decreased by different amounts for different iterations.

Once the stimulation rate is decreased below the minimum rate, the method 900 can proceed to block 912 with setting the stimulation rate back to the initial rate, and then to block 914 with increasing the stimulation rate. The stimulation rate can be increased by a predetermined (e.g., fixed) amount. For instance, in embodiments where the stimulation rate is a pacing rate, the pacing rate can be increased by 1 BPM, 2 BPM, 3 BPM, 4 BPM, 5 BPM, 10 BPM, or 20 BPM. The magnitude of the increase can be sufficiently large to affect the physiological motion (e.g., by shifting one or more spectral peaks of the motion by at least 0.1 Hz, 0.25 Hz, 0.5 Hz, 1 Hz, 2 Hz, 5 Hz, or 10 Hz), but not so large as to be noticeable by the patient and/or cause detrimental effects (e.g., harm and/or discomfort).

At block 916, the method 900 can include determining whether the increased stimulation rate is above a maximum rate. The maximum rate can be a predetermined value based on considerations of patient safety (e.g., increasing the rate above the maximum rate may cause harm and/or discomfort), therapeutic efficacy (e.g., increasing the rate above the maximum rate may result in loss of therapeutic benefit and/or otherwise be therapeutically inappropriate), device limitations, etc. The appropriate maximum rate can be a patient-specific rate based on the particular characteristics of the patient, or can be a generic rate that is applicable to broader category of patients (e.g., patients of a particular age, weight, etc.) or to all patients. In the context of pacing rate, the maximum rate can be within 1 BPM, 2 BPM, 3 BPM, 4 BPM, 5 BPM, 10 BPM, or 20 BPM of the initial pacing rate (e.g., a SIR output by a rate-responsive pacing function).

If the increased stimulation rate is not greater than the maximum rate, the method 900 can continue to block 918 with delivering the stimulation signal at the increased stimulation rate and measuring the resulting power output of the energy harvesting mechanism, e.g., as described above with respect to block 904. The processes of blocks 914, 916, and 918 can be repeated to incrementally increase the stimulation rate and determine the resulting power output, until the maximum rate is reached. The stimulation rate can be increased by the same amount for each iteration, or can be increased by different amounts for different iterations.

Once the stimulation rate has been increased above the maximum rate, the method 900 can continue to block 920 with selecting a stimulation rate that is associated with a desired power output of the energy harvesting mechanism. The stimulation rate can be selected according to any suitable set of criteria. In some embodiments, for example, the selected stimulation rate is the tested rate that achieved the highest power output, such as the highest net power and/or net current to the power source, highest absolute power output, highest absolute current output, etc. As another example, the selected stimulation rate can be the tested rate that was closest to the initial rate while still producing a power output above a predetermined threshold. In a further example, lower stimulation rates may be prioritized over higher stimulation rates (or vice-versa), as long as the power output of the selected rate is above the threshold.

The method 900 can be modified in many different ways. In some embodiments, the method 900 is performed without testing the entire range of stimulation rates between the minimum rate and the maximum rate. For instance, if the results obtained during the processes of blocks 906, 908, and 910 show that the power output is trending in an unfavorable direction with decreasing stimulation rates (e.g., power output is decreasing sharply), the method 900 can stop testing decreasing stimulation rates even before the minimum rate has been reached, and can instead proceed directly to blocks 912 and 914 with testing increasing stimulation rates. Similarly, if the results obtained during the processes of blocks 914, 916, and 918 show that the power output is trending in an unfavorable direction with increasing stimulation rates, the method 900 can stop testing increasing stimulation rates even before the maximum rate has been reached, and can instead proceed directly to block 920. This approach can reduce the total time needed to determine the appropriate stimulation rate. Testing can also be terminated early due to other considerations, such as if detrimental effects are observed, if the patient's condition (e.g., activity level) changes significantly, etc.

Moreover, although FIG. 9 illustrates an embodiment in which lower stimulation rates are tested before higher stimulation rates, the method 900 can alternatively include performing the processes of blocks 914, 916, and 918 before the processes of blocks 906, 908, and 910 to test higher stimulation rates before lower stimulation rates. Moreover, testing can instead be performed in a unidirectional manner, e.g., the initial stimulation rate is the maximum rate and the method 900 involves decreasing the stimulation rate until the minimum rate is reached, or the initial stimulation rate is the minimum rate and the method 900 involves increasing the stimulation rate until the maximum rate is reached. Optionally, testing can be performed in a random order or any other suitable order within a particular range of stimulation rates.

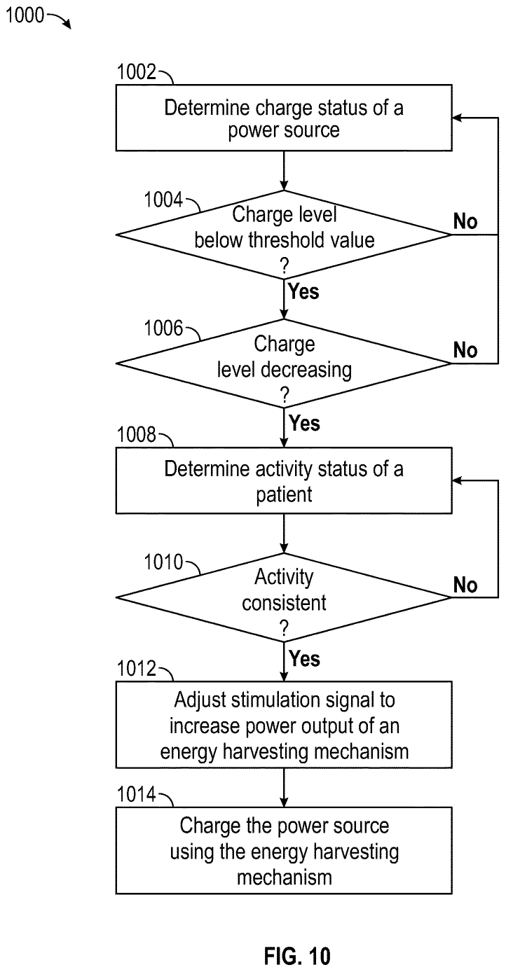

FIG. 10 is a flow diagram illustrating a method 1000 for powering an implantable device, in accordance with embodiments of the present technology. The method 1000 can be performed in combination with any of the other methods described herein, such as the method 700 of FIG. 7 and/or the method 900 of FIG. 9. The method 1000 can be performed using any of the systems and devices described herein, such as any of the devices of FIGS. 1-5. In some embodiments, some or all of the processes of the method 1000 are implemented as computer-readable instructions (e.g., program code) that are configured to be executed by one or more processors (e.g., processing circuitry 414 of the device 400 of FIG. 4).

The method 1000 can begin at block 1002 with determining charge status of a power source. The power source can be a rechargeable battery onboard the implantable device, as described elsewhere herein. The charge status can include parameters such as the charge level; whether the charge level is increasing, decreasing, or remaining constant; the rate at which the charge level is increasing or decreasing; the estimated remaining battery life (e.g., time to 0% charge); etc. The charge status can be determined using power management circuitry and/or other suitable electronics that are electrically coupled to the power source.

At block 1004, the method 1000 can include determining whether the charge level is below a threshold value. The threshold value can correlate to full charge, low charge, or any other suitable charge level. For example, the threshold value can be 100%, 90%, 80%, 70%, 60%, 50%, 40%, 30%, 20%, 10%, or 5% of the full charge level. If the charge level is not below the threshold value, this can indicate that the power source is not in need of charging, and the method 1000 can return to block 1002 to continue monitoring the charge status of the power source.

If the charge level is below the threshold value, the method 1000 can continue to block 1006 with determining whether the charge level is decreasing. A decreasing charge level can indicate that the power source is at risk of running out of power in the near future and thus should be charged as soon as possible. Conversely, a constant or increasing charge level can indicate that the power source is not at imminent risk of running out of power, such that charging is not needed and the method 1000 can return to block 1002 to continue monitoring the power source. In some embodiments, the process of block 1006 further includes determining the rate at which the charge level is decreasing to estimate the remaining battery life. If the remaining battery life is below a predetermined value (e.g., less than or equal to 24 hours, 12 hours, 10 hours, 5 hours, or 1 hour), this can also indicate that charging should be performed immediately. If the remaining battery life is above the predetermined value, charging can be delayed or may be unnecessary.

If the charge level is decreasing, the method 1000 can proceed to block 1008 with determining the activity status of the patient. The activity status can include information regarding the current activity level of the patient (e.g., whether the patient is engaging in low, moderate, or strenuous activity; activity counts produced by an activity sensor), the type of activity the patient is engaged in (e.g., sleeping, resting, walking, running), the variability of the patient's activity over time, the amount of time the patient has spent at the current activity level, trends in activity level, etc. The activity status can be determined using one or more activity sensors onboard the implantable device, such as a motion sensor (e.g., IMU, accelerometer, gyroscope), position sensor, and the like. Alternatively or in combination, the activity status can be determined using another device that is communicably coupled to the implantable device. For instance, patient activity can be monitored using a wearable device (e.g., smartwatch), a mobile device (e.g., smartphone), and/or other internal or external activity sensor. The activity data generated by the other device can be transmitted to the implantable device (e.g., directly or via an intermediary, such as the external device 110 of FIG. 1).

At block 1010, the method 1000 can include determining whether the patient's activity is consistent, based on the determined activity status. The process of block 1010 can include determining whether the patient's activity is currently consistent and/or is predicted to remain consistent within the near future (e.g., for the next 5 minutes, 10 minutes, 30 minutes, 60 minutes, or more). In some embodiments, it may be advantageous to charge the power source while the patient is exhibiting consistent activity and the physiological motion of the patient is expected to remain stable. Moreover, in the context of cardiac pacing, changes in patient activity may necessitate changes to the pacing rate, which can interfere with rate control for energy harvesting purposes.

The consistency of the patient's activity can be determined by in various ways. For example, the process of block 1010 can include assessing whether the patient has been at the same or a similar activity level for a predetermined period of time (e.g., at least 1 minute, 2 minutes, 5 minutes, 10 minutes, 30 minutes, or 60 minutes). The patient's activity can be considered consistent if an activity metric (e.g., activity count) does not vary by more than 20%, 10%, 5%, 2%, or 1% from an average of the activity metric over the time period. Alternatively or in combination, the process of block 1010 can include evaluating whether the patient is engaged in a type of activity that is likely to remain consistent for a prolonged period of time, such as sleeping, resting, or moderate walking. In some embodiments, the patient's activity is more likely to remain consistent if the patient's current activity level is low or moderate, and is less likely to remain consistent if the patient's current activity level is high.

If the patient's activity is not consistent, the method 1000 can return to block 1008 with monitoring the patient's activity status, and charging of the power source can be delayed until the patient activity becomes consistent.

If the patient's activity is consistent, the method 1000 can continue to block 1012 with adjusting a stimulation signal to increase the power output of an energy harvesting mechanism, and then to block 1014 with charging the power source using the energy harvesting mechanism. The processes of blocks 1012 and 1014 can be performed in accordance with the techniques of the method 700 of FIG. 7.

The method 1000 can be modified in many different ways. For example, the method 1000 can include additional processes not shown in FIG. 10. In some embodiments, the method 1000 can further include determining a second charge status of the power source, after the processes of blocks 1012 and 1014. The second charge status can indicate whether the power source has reached a desired charge level, such as fully charged, sufficiently charged (e.g., at least 50%, 60%, 70%, 75%, 80%, 90%, or 95% of full charge), and/or an increased charge level relative to the initial charge level in block 1004. If the power source has been charged to the desired charge level, the stimulation signal can be reverted to an initial and/or non-adjusted signal (e.g., a SIR or other baseline pacing rate). The method 1000 can then return to block 1002 at a later time point to determine whether further charging of the power source is appropriate.

As another example, some of the processes of the method 1000 can be omitted, such as the process of block 1006, so that charging is performed as long as the charge level of the power source is sufficiently low. As another example, the processes of blocks 1008 and 1010 can be omitted, such that charging can be performed regardless of the patient's activity level. These approaches may enhance safety by ensuring that charging occurs whenever the power source is at risk of depletion.

Although certain embodiments of the present technology are described in connection with determining and enhancing the power output of an energy harvesting mechanism, the embodiments herein can alternatively or additionally include determining and enhancing the energy output of the energy harvesting mechanism. The energy output of the energy harvesting mechanism can be related to the power output, and can be determined by measuring and integrating the amount of power produced by the energy harvesting mechanism over a predetermined period of time, and/or by measuring and integrating the net power into the power source over a predetermined period of time.

### EXAMPLES

The following examples are included to further describe some aspects of the present technology, and should not be used to limit the scope of the technology.
1. A device comprising:
   an energy harvesting mechanism configured to produce energy from physiological motion of a patient;
   one or more electrodes configured to deliver electrical stimulation to the patient;
   a power source operably coupled to the energy harvesting mechanism and the one or more electrodes;
   processing circuitry; and
   a memory operably coupled to the processing circuitry and storing instructions that, when executed by the processing circuitry, cause the device to perform operations comprising:
      determining a power output of the energy harvesting mechanism;
      determining a stimulation signal configured to adjust the physiological motion to increase the power output of the energy harvesting mechanism;
      delivering the stimulation signal to the patient using the one or more electrodes; and
      charging the power source using the energy harvesting mechanism.
2. The device of Example 1, wherein the stimulation signal comprises a pacing signal, the physiological motion comprises cardiac motion, and determining the stimulation signal comprises determining a pacing rate for the pacing signal.
3. The device of Example 2, wherein the pacing rate for the pacing signal is determined by:
   delivering the pacing signal at a plurality of pacing rates,
   measuring the power output of the energy harvesting mechanism for each pacing rate, and
   selecting a pacing rate of the plurality of pacing rates that is associated with a desired power output.
4. The device of Example 3, wherein the plurality of pacing rates are within a predetermined range of an initial pacing rate, and the initial pacing rate is determined based on an activity level of the patient.
5. The device of Example 4, wherein the plurality of pacing rates comprise a first set of pacing rates less than the initial pacing rate, and a second set of pacing rates greater than the initial pacing rate.
6. The device of any one of Examples 1 to 5, wherein the stimulation signal is configured to adjust the physiological motion such that a frequency component of the physiological motion overlaps or approaches a resonant frequency of the energy harvesting mechanism.
7. The device of Example 6, wherein the resonant frequency of the energy harvesting mechanism is within a range from 10 Hz to 30 Hz.
8. The device of Example 6 or 7, wherein the stimulation signal is configured to adjust the physiological motion so that the frequency component of the physiological motion is within 5 Hz of the resonant frequency of the energy harvesting mechanism.
9. The device of any one of Examples 1 to 8, wherein the stimulation signal is determined by:
   delivering a plurality of stimulation signals to the patient, wherein the plurality of stimulation signals differ from each other with respect to at least one stimulation parameter,
   measuring a power output of the energy harvesting mechanism for each stimulation signal, and
   selecting one of the plurality of stimulation signals, based on the measured power outputs.
10. The device of Example 9, wherein the at least one stimulation parameter comprises a stimulation rate.
11. The device of Example 9 or 10, wherein the selected one of the plurality of stimulation signals is a stimulation signal associated with the highest power output.
12. The device of any one of Examples 1 to 11, wherein the energy harvesting mechanism comprises an elongate piezoelectric member configured to deform in response to the physiological motion.
13. The device of any one of Examples 1 to 12, wherein the power output of the energy harvesting mechanism is determined by measuring one or more of a net power or a net current into the power source.
14. The device of any one of Examples 1 to 13, wherein the operations further comprise:
   monitoring a charge level of the power source, and
   if (a) the charge level is below a threshold value, (b) the charge level is decreasing, or both (a) and (b), performing the processes of determining the stimulation signal, delivering the stimulation signal, and charging the power source.
15. The device of any one of Examples 1 to 14, wherein the operations further comprise:
   monitoring activity of the patient using an activity sensor, and
   if the activity of the patient is consistent, performing the processes of determining the stimulation signal, delivering the stimulation signal, and charging the power source.
16. The device of any one of Examples 1 to 15, wherein the stimulation signal is determined by:
   determining a pacing rate based on data from an activity sensor, and
   determining a modification to the pacing rate to increase the power output of the energy harvester.
17. The device of any one of Examples 1 to 16, wherein the stimulation signal is determined by:
   determining a pacing rate based on a rate-responsive pacing function, and
   determining a modification to the pacing rate to increase the power output of the energy harvester.
18. A method comprising:
   measuring, via processing circuitry, power output of an energy harvester of an implantable device, wherein the energy harvester is configured to generate energy from physiological motion of a patient;
   determining, via the processing circuitry, a stimulation signal configured to adjust the physiological motion to increase the power output of the energy harvester;
   applying the stimulation signal to the patient using the implantable device; and
   recharging a power source of the implantable device using the energy harvester.
19. The method of Example 18, wherein determining the stimulation signal comprises determining a pacing rate for a pacing signal configured to set a cardiac rhythm of the patient.
20. The method of Example 19, wherein determining the pacing rate for the pacing signal comprises:
   delivering the pacing signal at a plurality of pacing rates,
   measuring the power output of the energy harvester for each pacing rate, and
   selecting a pacing rate of the plurality of pacing rates that is associated with a desired power output.
21. The method of Example 20, further comprising identifying a baseline pacing rate based on activity of the patient, wherein the plurality of pacing rates are within a predetermined range of the baseline pacing rate.
22. The method of Example 21, wherein the plurality of pacing rates comprise a first set of pacing rates less than the baseline pacing rate, and a second set of pacing rates greater than the baseline pacing rate.
23. The method of any one of Examples 19 to 22, wherein the pacing signal is configured to treat a cardiac condition of the patient.
24. The method of any one of Examples 18 to 23, wherein the stimulation signal is configured to adjust the physiological motion such that a spectral peak of a frequency spectrum of the physiological motion overlaps or approaches a resonant frequency of the energy harvester.
25. The method of Example 24, wherein the resonant frequency of the energy harvester is within a range from 10 Hz to 30 Hz.
26. The method of Example 24 or 25, wherein the stimulation signal is configured to adjust the physiological motion so that a peak frequency of the spectral peak is within 5 Hz of the resonant frequency of the energy harvester.
27. The method of any one of Examples 18 to 26, wherein determining the stimulation signal comprises:
   delivering a plurality of stimulation signals to the patient, wherein the plurality of stimulation signals differ from each other with respect to at least one stimulation parameter,
   measuring a power output of the energy harvester for each stimulation signal, and
   selecting one of the plurality of stimulation signals, based on the measured power outputs.
28. The method of Example 27, wherein the at least one stimulation parameter comprises a stimulation rate.
29. The method of Example 27 or 28, wherein the selected one of the plurality of stimulation signals is a stimulation signal associated with the highest power output.
30. The method of any one of Examples 18 to 29, measuring the power output of the energy harvester comprises measuring one or more of a net power or a net current into the power source.
31. The method of any one of Examples 18 to 30, further comprising:
   monitoring a charge level of the power source, and
   if (a) the charge level is below a threshold value, (b) the charge level is decreasing, or both (a) and (b), performing the processes of determining the stimulation signal, delivering the stimulation signal, and charging the power source.
32. The method of any one of Examples 18 to 31, further comprising:
   monitoring activity of the patient using an activity sensor, and
   if the activity of the patient is consistent, performing the processes of determining the stimulation signal, delivering the stimulation signal, and charging the power source.
33. The method of any one of Examples 18 to 32, wherein determining the stimulation signal comprises:
   determining a pacing rate based on an activity level of the patient, and
   determining a modification to the pacing rate using a transfer function.
34. The method of any one of Examples 18 to 33, wherein determining the stimulation signal comprises:
   determining a pacing rate based on a rate-responsive pacing function, and
   determining a modification to the pacing rate using a transfer function.
35. A non-transitory computer-readable storage medium comprising instructions that, when executed by one or more processors of an implantable device comprising an energy harvesting mechanism, cause the implantable device to perform operations comprising:
   determining a stimulation signal configured to adjust physiological motion of a patient to increase a power output of the energy harvesting mechanism relative to a baseline power output of the energy harvesting mechanism;
   delivering the stimulation signal to the patient; and
   charging a power source of the implantable device using the energy harvesting mechanism.

### Conclusion

Although many of the embodiments are described above with respect to systems, devices, and methods for cardiac pacing, the technology is applicable to other applications and/or other approaches, such as other therapies involving implantable devices. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-10.

The embodiments of the present technology can be implemented, at least in part, in hardware, software, firmware or any combination thereof. For example, various embodiments can be implemented within one or more processors, including one or more microprocessors, DSPs, ASICs, FPGAs, or any other equivalent integrated or discrete logic circuitry, as well as any combinations of such components, embodied in programmers (e.g., physician or patient programmers), stimulators, or other devices. The terms “processor” or “processing circuitry” may generally refer to any of the foregoing logic circuitry, alone or in combination with other logic circuitry, or any other equivalent circuitry.

The various processes described herein can be partially or fully implemented using program code including instructions executable by one or more processors of a computing system for implementing specific logical functions or steps in the process. The program code can be stored on any type of computer-readable medium, such as a storage device including a disk or hard drive. Computer-readable media containing code, or portions of code, can include any appropriate media known in the art, such as non-transitory computer-readable storage media. Computer-readable media can include volatile and non-volatile, removable and non-removable media implemented in any method or technology for storage and/or transmission of information, including, but not limited to, random-access memory (RAM), read-only memory (ROM), electrically erasable programmable read-only memory (EEPROM), flash memory, or other memory technology; compact disc read-only memory (CD-ROM), digital video disc (DVD), or other optical storage; magnetic cassettes, magnetic tape, magnetic disk storage, or other magnetic storage devices; solid state drives (SSD) or other solid state storage devices; or any other medium which can be used to store the desired information and which can be accessed by a system device.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded.

To the extent any materials incorporated herein by reference conflict with the present disclosure, the present disclosure controls.

It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

The following examples are a non-limiting list of clauses in accordance with one or more techniques of this disclosure.

Example 1. A device comprising: an energy harvesting mechanism configured to produce energy from physiological motion of a patient; one or more electrodes configured to deliver electrical stimulation to the patient; a power source operably coupled to the energy harvesting mechanism and the one or more electrodes; processing circuitry; and a memory operably coupled to the processing circuitry and storing instructions that, when executed by the processing circuitry, cause the device to perform operations comprising: determining a power output of the energy harvesting mechanism; determining a stimulation signal configured to adjust the physiological motion to increase the power output of the energy harvesting mechanism; delivering the stimulation signal to the patient using the one or more electrodes; and charging the power source using the energy harvesting mechanism.

Example 2. The device of Example 1, wherein the stimulation signal comprises a pacing signal, the physiological motion comprises cardiac motion, and determining the stimulation signal comprises determining a pacing rate for the pacing signal.

Example 3. The device of Example 2, wherein the pacing rate for the pacing signal is determined by: delivering the pacing signal at a plurality of pacing rates, measuring the power output of the energy harvesting mechanism for each pacing rate, and selecting a pacing rate of the plurality of pacing rates that is associated with a desired power output.

Example 4. The device of Example 3, wherein the plurality of pacing rates are within a predetermined range of an initial pacing rate, and the initial pacing rate is determined based on an activity level of the patient.

Example 5. The device of Example 4, wherein the plurality of pacing rates comprise a first set of pacing rates less than the initial pacing rate, and a second set of pacing rates greater than the initial pacing rate.

Example 6. The device of any one of Examples 1 to 5, wherein the stimulation signal is configured to adjust the physiological motion such that a frequency component of the physiological motion overlaps or approaches a resonant frequency of the energy harvesting mechanism.

Example 7. The device of Example 6, wherein the resonant frequency of the energy harvesting mechanism is within a range from 10 Hz to 30 Hz.

Example 8. The device of Example 6 or 7, wherein the stimulation signal is configured to adjust the physiological motion so that the frequency component of the physiological motion is within 5 Hz of the resonant frequency of the energy harvesting mechanism.

Example 9. The device of any one of Examples 1 to 8, wherein the stimulation signal is determined by: delivering a plurality of stimulation signals to the patient, wherein the plurality of stimulation signals differ from each other with respect to at least one stimulation parameter, measuring a power output of the energy harvesting mechanism for each stimulation signal, and selecting one of the plurality of stimulation signals, based on the measured power outputs.

Example 10. The device of Example 9, wherein the at least one stimulation parameter comprises a stimulation rate.

Example 11. The device of Example 9 or 10, wherein the selected one of the plurality of stimulation signals is a stimulation signal associated with the highest power output.

Example 12. The device of any one of Examples 1 to 11, wherein the energy harvesting mechanism comprises an elongate piezoelectric member configured to deform in response to the physiological motion.

Example 13. The device of any one of Examples 1 to 12, wherein the power output of the energy harvesting mechanism is determined by measuring one or more of a net power or a net current into the power source.

Example 14. The device of any one of Examples 1 to 13, wherein the operations further comprise: monitoring a charge level of the power source, and if (a) the charge level is below a threshold value, (b) the charge level is decreasing, or both (a) and (b), performing the processes of determining the stimulation signal, delivering the stimulation signal, and charging the power source.

Example 15. The device of any one of Examples 1 to 14, wherein the operations further comprise: monitoring activity of the patient using an activity sensor, and if the activity of the patient is consistent, performing the processes of determining the stimulation signal, delivering the stimulation signal, and charging the power source.

Example 16. The device of any one of Examples 1 to 15, wherein the stimulation signal is determined by: determining a pacing rate based on data from an activity sensor, and determining a modification to the pacing rate to increase the power output of the energy harvester.

Example 17. The device of any one of Examples 1 to 16, wherein the stimulation signal is determined by: determining a pacing rate based on a rate-responsive pacing function, and determining a modification to the pacing rate to increase the power output of the energy harvester.

Example 18. A method comprising: measuring, via processing circuitry, power output of an energy harvester of an implantable device, wherein the energy harvester is configured to generate energy from physiological motion of a patient; determining, via the processing circuitry, a stimulation signal configured to adjust the physiological motion to increase the power output of the energy harvester; applying the stimulation signal to the patient using the implantable device; and recharging a power source of the implantable device using the energy harvester.

Example 19. The method of Example 18, wherein determining the stimulation signal comprises determining a pacing rate for a pacing signal configured to set a cardiac rhythm of the patient.

Example 20. The method of Example 19, wherein determining the pacing rate for the pacing signal comprises: delivering the pacing signal at a plurality of pacing rates, measuring the power output of the energy harvester for each pacing rate, and selecting a pacing rate of the plurality of pacing rates that is associated with a desired power output.

Example 21. The method of Example 20, further comprising identifying a baseline pacing rate based on activity of the patient, wherein the plurality of pacing rates are within a predetermined range of the baseline pacing rate.

Example 22. The method of Example 21, wherein the plurality of pacing rates comprise a first set of pacing rates less than the baseline pacing rate, and a second set of pacing rates greater than the baseline pacing rate.

Example 23. The method of any one of Examples 19 to 22, wherein the pacing signal is configured to treat a cardiac condition of the patient.

Example 24. The method of any one of Examples 18 to 23, wherein the stimulation signal is configured to adjust the physiological motion such that a spectral peak of a frequency spectrum of the physiological motion overlaps or approaches a resonant frequency of the energy harvester.

Example 25. The method of Example 24, wherein the resonant frequency of the energy harvester is within a range from 10 Hz to 30 Hz.

Example 26. The method of Example 24 or 25, wherein the stimulation signal is configured to adjust the physiological motion so that a peak frequency of the spectral peak is within 5 Hz of the resonant frequency of the energy harvester.

Example 27. The method of any one of Examples 18 to 26, wherein determining the stimulation signal comprises: delivering a plurality of stimulation signals to the patient, wherein the plurality of stimulation signals differ from each other with respect to at least one stimulation parameter, measuring a power output of the energy harvester for each stimulation signal, and selecting one of the plurality of stimulation signals, based on the measured power outputs.

Example 28. The method of Example 27, wherein the at least one stimulation parameter comprises a stimulation rate.

Example 29. The method of Example 27 or 28, wherein the selected one of the plurality of stimulation signals is a stimulation signal associated with the highest power output.

Example 30. The method of any one of Examples 18 to 29, measuring the power output of the energy harvester comprises measuring one or more of a net power or a net current into the power source.

Example 31. The method of any one of Examples 18 to 30, further comprising: monitoring a charge level of the power source, and if (a) the charge level is below a threshold value, (b) the charge level is decreasing, or both (a) and (b), performing the processes of determining the stimulation signal, delivering the stimulation signal, and charging the power source.

Example 32. The method of any one of Examples 18 to 31, further comprising: monitoring activity of the patient using an activity sensor, and if the activity of the patient is consistent, performing the processes of determining the stimulation signal, delivering the stimulation signal, and charging the power source.

Example 33. The method of any one of Examples 18 to 32, wherein determining the stimulation signal comprises: determining a pacing rate based on an activity level of the patient, and determining a modification to the pacing rate using a transfer function.

Example 34. The method of any one of Examples 18 to 33, wherein determining the stimulation signal comprises: determining a pacing rate based on a rate-responsive pacing function, and determining a modification to the pacing rate using a transfer function.

Example 35. A non-transitory computer-readable storage medium comprising instructions that, when executed by one or more processors of an implantable device comprising an energy harvesting mechanism, cause the implantable device to perform operations comprising: determining a stimulation signal configured to adjust physiological motion of a patient to increase a power output of the energy harvesting mechanism relative to a baseline power output of the energy harvesting mechanism; delivering the stimulation signal to the patient; and charging a power source of the implantable device using the energy harvesting mechanism.

1. A device comprising:
an energy harvesting mechanism configured to produce energy from physiological motion of a patient;
one or more electrodes configured to deliver electrical stimulation to the patient;
a power source operably coupled to the energy harvesting mechanism and the one or more electrodes;
processing circuitry; and
a memory operably coupled to the processing circuitry and storing instructions that, when executed by the processing circuitry, cause the device to perform operations comprising:
determining a power output of the energy harvesting mechanism;
determining a stimulation signal configured to adjust the physiological motion to increase the power output of the energy harvesting mechanism;
delivering the stimulation signal to the patient using the one or more electrodes; and
charging the power source using the energy harvesting mechanism.

2. The device of claim 1, wherein the stimulation signal comprises a pacing signal, the physiological motion comprises cardiac motion, and determining the stimulation signal comprises determining a pacing rate for the pacing signal.

3. The device of claim 2, wherein the pacing rate for the pacing signal is determined by:
delivering the pacing signal at a plurality of pacing rates,
measuring the power output of the energy harvesting mechanism for each pacing rate, and
selecting a pacing rate of the plurality of pacing rates that is associated with a desired power output.

4. The device of claim 3, wherein the plurality of pacing rates are within a predetermined range of an initial pacing rate, and the initial pacing rate is determined based on an activity level of the patient.

5. The device of claim 4, wherein the plurality of pacing rates comprise a first set of pacing rates less than the initial pacing rate, and a second set of pacing rates greater than the initial pacing rate.

6. The device of a claim 1, wherein the stimulation signal is configured to adjust the physiological motion such that a frequency component of the physiological motion overlaps or approaches a resonant frequency of the energy harvesting mechanism.

7. The device of claim 6, wherein the resonant frequency of the energy harvesting mechanism is within a range from 10 Hz to 30 Hz.

8. The device of claim 6, wherein the stimulation signal is configured to adjust the physiological motion so that the frequency component of the physiological motion is within 5 Hz of the resonant frequency of the energy harvesting mechanism.

9. The device of claim 1, wherein the stimulation signal is determined by:
delivering a plurality of stimulation signals to the patient, wherein the plurality of stimulation signals differ from each other with respect to at least one stimulation parameter,
measuring a power output of the energy harvesting mechanism for each stimulation signal, and
selecting one of the plurality of stimulation signals, based on the measured power outputs.

10. The device of claim 9, wherein the at least one stimulation parameter comprises a stimulation rate.

11. The device of claim 9, wherein the selected one of the plurality of stimulation signals is a stimulation signal associated with the highest power output.

12. The device of claim 1, wherein the energy harvesting mechanism comprises an elongate piezoelectric member configured to deform in response to the physiological motion.

13. The device of claim 1, wherein the power output of the energy harvesting mechanism is determined by measuring one or more of a net power or a net current into the power source.

14. The device of claim 1, wherein the operations further comprise:
monitoring a charge level of the power source, and
if (a) the charge level is below a threshold value, (b) the charge level is decreasing, or both (a) and (b), performing the processes of determining the stimulation signal, delivering the stimulation signal, and charging the power source.

15. The device of as claim 1, wherein the stimulation signal is determined by:
determining a pacing rate based on data from an activity sensor, and
determining a modification to the pacing rate to increase the power output of the energy harvester.

16. A method comprising:
measuring, via processing circuitry, power output of an energy harvester of an implantable device, wherein the energy harvester is configured to generate energy from physiological motion of a patient;
determining, via the processing circuitry, a stimulation signal configured to adjust the physiological motion to increase the power output of the energy harvester;
applying the stimulation signal to the patient using the implantable device; and
recharging a power source of the implantable device using the energy harvester.

17. The method of claim 16, wherein determining the stimulation signal comprises determining a pacing rate for a pacing signal configured to set a cardiac rhythm of the patient.

18. The method of claim 17, wherein wherein determining the pacing rate for the pacing signal comprises:
delivering the pacing signal at a plurality of pacing rates, measuring the power output of the energy harvester for each pacing rate, and
selecting a pacing rate of the plurality of pacing rates that is associated with a desired power output.

19. The method of claim 18, further comprising identifying a baseline pacing rate based on activity of the patient, wherein the plurality of pacing rates are within a predetermined range of the baseline pacing rate.

20. The method of claim 15, wherein the stimulation signal is configured to adjust the physiological motion such that a frequency component of the physiological motion overlaps or approaches a resonant frequency of the energy harvester.
